SEQUENCE LISTING (29 sequences)

SEQ ID NO: 1 - DNA, 24 bp, Human immunodeficiency virus
gcccctaggaaaaagggctgttgg

SEQ ID NO: 2 - DNA, 33 bp, Human immunodeficiency virus
catgagaaatatcacagtaattggagagcaatg

SEQ ID NO: 3 - DNA, 30 bp, Human immunodeficiency virus
aatgtggaaaggaaggacaccaaatgaaag

SEQ ID NO: 4 - DNA, 29 bp, Human immunodeficiency virus
ctcataaccgttcggtggacctaaggact

SEQ ID NO: 5 - DNA, 19 bp, Human immunodeficiency virus
gagagcttcaggtttgggg

SEQ ID NO: 6 - DNA, 18 bp, Human immunodeficiency virus
aattgggcctgaaaatcc

SEQ ID NO: 7 - DNA, 20 bp, Human immunodeficiency virus
cctccattcctttggatggg

SEQ ID NO: 8 - DNA, 19 bp, Human immunodeficiency virus
cactctttggcaacgaccc

SEQ ID NO: 9 - DNA, 17 bp, Human immunodeficiency virus
ctcccactcaggaatcc

SEQ ID NO: 10 - DNA, 21 bp, Human immunodeficiency virus
cttcccagaagtcttgagttc

SEQ ID NO: 11 - DNA, 20 bp, Human immunodeficiency virus
gggtcataatacactccatg

SEQ ID NO: 12 - DNA, 19 bp, Human immunodeficiency virus
ggaatattgctggtgatcc

SEQ ID NO: 13 - DNA, 21 bp, Human immunodeficiency virus
cagaccagagccaacagcccc

SEQ ID NO: 14 - DNA, 20 bp, Human immunodeficiency virus
ggtacagtattagtaggacc

SEQ ID NO: 15 - DNA, 20 bp, Human immunodeficiency virus
gtactggatgtgggtgatgc

SEQ ID NO: 16 - DNA, 19 bp, Human immunodeficiency virus
gtgggaaaattgaattggg

SEQ ID NO: 17 - DNA, 19 bp, Human immunodeficiency virus
gtactgtccatttatcagg

SEQ ID NO: 18 - DNA, 31 bp, Human immunodeficiency virus
ctaactggtaccataatttcactaagggagg

SEQ ID NO: 19 - DNA, 20 bp, Human immunodeficiency virus
cattgtttaacttttgggcc

SEQ ID NO: 20 - DNA, 18 bp, Human immunodeficiency virus
gataaaacctccaattcc

SEQ ID NO: 21 - DNA, 2205 bp, Human immunodeficiency virus
gaattctgtggtttcatttactctaaacctgtgattcctctgaattattttcattttaaagaaattgtatttgttaaatatgtactacaaacttagtagttggaagggttaatttactccaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacacccggagttttacaaagactgctgacacagaagggactttccgctgggactttccactggggcgttccaggaggtgtggtctgggcgggactgggagtggtcaaccctcagatgcggcatataagccgctgcttttcgcttgtactgggtctctctaggtagaccagatctgagcctgggagctctctggctatctagggaacccactgcttaagcctcaataaagcttgccttgagtgctctgagcagtgtgtgcccgtctattgtgtgactctggtaactagagatccctcagacccttttggtagtgtggaaaatctctagcagtggcgcccgaacagggacttgaaagcgaaagtaagaccagagaagatcctctagacgcaggactcggcttgctgaagtgcactcggcaagaggcgagagcggcgactggtgagtacgccaattttatttgactagcggaggctagaaggagagagatgggtgcgagagcgtcaatattaagagggggaaaattagataaatgggaaagaattaggttaaggccagggggaaagaaacactatatgctaaaacacctagtatgggcaagcagggagctggaaagattcgcactcaaccctggccttttagagacagcagaaggctgtaaacaaataataaaacagctacaaccagctcttcagacaggaacagaggaacttaaatcattacacaacacagtagcaactctctattgtgtacatgcagggatagaagtacgagacaccaaagaagccttagacaagatagaggaagaacaaaacaaaattcagcaaaaaacacaacaggcaaaagaggctgacgggaaggtcagtcaaaattatcctatagtgcagaatctccaagggcaaatggtacaccaggccatatcacctagaactttgaatgcatgggtaaaagtaatagaggagaaggcttttagcccagaggtaatacccatgtttacagcattatcagaaggagccaccccacaagacttaaacaccatgttaaatacagtggggggacatcaagcagccatgcaaatgttaaaagataccatcaatgaagaggctgcagaatgggatagattacatccaatccatgcagggcctattgcaccaggccaaatgagagaaccaaggggaagtgacatagcaggaactactagtagccttcaggaacaaatagcatggatgacagggaacccacctgttccagtgggagacatctataaaagatggataattctggggttaaataaaatagtaagaatgtatagccctgttagcattttggacataagacaagggccaaaggaaccctttagagactatgtagaccggttctttaaaactttaagagctgaacaagctacacaagatgtaaaaaattggatgacagacaccttgttggtccaaaatgcgaatccagattgtaagaccattttaagagcattaggaccaggggcttcattagaagagatgatgacagcatgtcagggagtgggaggacctggccacaaagcaagagtgttggctgaggcaatgagccaagcaaacagtaccatactgatgcagagaagcaattttaaaggctctaaaagaattgttaaatgtttcaactgtggcaaggaggggcacatagccaaaaattgcagggcccctaggaaaaaaggctgttggaaatgtggaaaggaaggtcacctggattcctgagtgggaatttgttaatacccctcccctagtaaaattatggtaccagctggggatcc

SEQ ID NO: 22 - DNA, 2612 bp, Human immunodeficiency virus
gaattcagtgggagacatctataaaagatggataattctggggttaaataaaatagtaagaatgtatagccctgttagcattttggacataagacaagggccaaaggaaccctttagagactatgtagaccggttctttaaaactttaagagctgaacaagctacacaagatgtaaaaaattggatgacagacaccttgttggtccaaaatgcgaatccagattgtaagaccattttaagagcattaggaccaggggcttcattagaagagatgatgacagcatgtcagggagtgggaggacctggccacaaagcaagagtgttggctgaggcaatgagccaagcaaacagtaccatactgatgcagagaagcaattttaaaggctctaaaagaattgttaaatgtttcaactgtggcaaggaggggcacatagccaaaaattgcagggcccctaggaaaaaaggctgttggaaatgtggaaaggaaggacaccaaatgaaagactgtactgagaggcaggctaattttttagggaaaatttggccttcccacaaggggaggccagggaatttcctccagagcagaccggagccaacagccccaccagcagagagcttcaggttcgaggagacaaccccagctccaaagcaggagccgaaagacagggaacccttaacttccctcaaatcactctttggcagcgacctcttgtctcaataagagtagggggccaaataaaagaggctctcttagacacaggagcagatgatacagtattagaagaagtaaatttgccaggaaaatggaaaccaaaaatgataggaggaattggaggttttatcaaagtaagacaatatgatcaaatacctatagaaatttgtggaaaaaaggctataggtacagtattagtgggacccacacctatcaacataattggaagaaatatgttgactcagcttggatgcacactaaattttccaatcagtcccattgaaactgtaccagtaaaattaaagccaggaatggatggcccaaaggttaaacaatggccattgacagaagagaaaataaaagcattaacagcaatttgtgatgaaatggagaaggaaggaaaaattacaaaaattgggcctgaaaatccatataacactccaatatttgctataaaaaagaaggacagtattaagtggagaaaattagtagatttcagggaactcaataaaagaactcaagatttttgggaagttcaattaggaataccacacccagcagggttaaaaaagaaaaaatcagtgacagtactggatgtgggggatgcatatttttcagttcctttatatgaagacttcaggaaatatactgcattcaccatacctagtataaacaatgaaacaccagggattaggtatcaatataatgtgcttccacagggatggaaaggatcaccagcaatattccagagtagcatgataagaatcttagagccctttagggcacaaaatccagaaatagtcatctatcaatatatggatgacttgtatgtaggatctgacttagaaatagggcaacatagagcaaaaatagaggagttaagagaacatctgttaaagtggggatttaccacaccagacaagaaacatcagaaggaacctccatttctttggatggggtatgaactccatcctgacaaatggacagtacagcctatacagctgccagaaaaggatagctggactgtcaatgatatacagaagttagtgggaaaattaaactgggcaagtcaaatttacccaggaattaaagtaaggcaactttgtaaactccttaggggggccaaagcactaacagacatagtaccactaactgaagaagcagaattagaattggcagaaaacagggaaattctaaaagaaccagtacatggagtatattatgacccatcaaaagacttgatagctgaaatacagaaacaggggcaggaccaatggacatatcaaatttaccaagaaccattcaaaaatctgaaaacagggaagtatgcaaaaaggaggactgcccacactaatgatgtaaaacagttgacagaggctgtgcagaaaatagccatggaaagcatagtaatatggggaaagactcctaaatttagattacctatccaaaaagaaacatgggagacatggtggacagactattggcaagccacctggattcctgagtgggaatttgttaatacccctcccctagtaaaattatggtaccagctggagaaagatcccatagcaggagtagaaactttctatgtagatggagcagctaatagggaaactaagttaggaaaagcagggtatgttactgacagaggaaggcagaaaattgtttctctaactgaaaccacaaatcagaagactgagttgcaagcaatttatctagctttgcaagattcaggatcagaagtaaacatagtaacagattcacagtatgcattagggatcattcaagcacaaccagataagagtgaatcagagttagttaaccaaataatcgaacagttaataaaaaaggaaagggtctatctgtcatgggtaccagcacataaaggaattggggatcc

SEQ ID NO: 23 - DNA, 3460 bp, Human immunodeficiency virus
gaattctttcaactgtggcaaggaggggcacatagccaaaaattgcagggcccctaggaaaaaaggctgttggaaatgtggaaaggaaggtcacctggattcctgagtgggaatttgttaatacccctcccctagtaaaattatggtaccagctggagaaagatcccatagcaggagtagaaactttctatgtagatggagcagctaatagggaaactaagttaggaaaagcagggtatgttactgacagaggaaggcagaaaattgtttctctaactgaaaccacaaatcagaagactgagttgcaagcaatttatctagctttgcaagattcaggatcagaagtaaacatagtaacagattcacagtatgcattagggatcattcaagcacaaccagataagagtgaatcagagttagttaaccaaataatcgaacagttaataaaaaaggaaagggtctatctgtcatgggtaccagcacataaaggaattggaggaaatgaacaagtagataaattagtaagtagtggaatcaggaaagtgctatttctagatggaatagataaagctcaagaagagcatgaaaagtatcacagcaattggagagcaatggccagtgactttaatctaccacccgtagtagcaaaagaaatagtagctagctgtgatcaatgtcagctaaaaggggaagccatgcatggacaagtagactgtagtccagggatatggcaattagattgtacacatttagaaggaaaaatcatcctggtagcagtccatgtagccagtggctacatagaagcagaggttattccagcagaaacaggacaagaaacagcatactttatactaaaattagcaggaagatggccagtcaaagtaatacatacagacaatggtagtaatttcaccagtgctgcagtcaaggcagcctgttggtgggcaggtatccaacaggaatttgggattccctacaatccccaaagtcagggagtagtagaatctatgaataaagaattaaagaaaattatagggcaggtaagagatcaagctgagcaccttaagacagcagtacaaatggcagtattcattcacaattttaaaagaaaaggggggattggggggtacagtgcaggggaaagaataatagacataatagcaacagacatacaaactaaagaattacaaaaacaaattataaaaattcaaaattttcgggtttattacagagacagcagagaccccatttggaaaggaccagccaaactactctggaaaggtgaaggggcagtagtaatacaagataatagtgacataaaggtagtaccacggaggaaagcaaaaatcattaaggactatggaaaacagatggcaggtgctgattgtgtggcaggtagacaggatgaagattagaacatggaatagtttagtaaaacaccatatgtatgtttcaaggagagctaaaggatggttttacagacatcattatgacagcagacatccaaaagtaagttcagaagtacacatcccattaggggaggctagattagtaataaaaacatattgggggttgcaaacaggagaaagagactggcatttgggtcatggagtctccatagaatggagattgagaagatataacacacaaatagaacctggcctggcagaccagctaatccatatgcattattttgattgttttgcagactctgccataaggaaagccatattaggacacatagttattcctaggtgtgactatcaagcaggacataataaggtaggatctctacaatacctggcactgacagcactgataaaaccaaaaaagataaagccacctctgcctagtattaagaaattagtagaggatagatggaacaatccccagaagatcaggggccgcagagggaaccatacaatgaatggacactagagcttctagaggaactcaagcaggaagctgtcagacactttcctagaccatggcttcatggcttaggacaatatgtctatgaaacatatggggatacttggacaggagtcgaagctataataagactactgcaacaactactgtttattcatttcagaattgggtgccagcatagcagaataggcattttgcgacagagaagagcaagaaatggagccagtagatcctaacctagagccctggaaccatccaggaagtcagcctaaaactgcttgcaatcaatgttattgtaaacgctgtagctatcattgtctagtttgctttcagaaaaaaggcttaggcatttcctatggcaggaagaagcggagacagcgacgaagcgctcctccaagcagtgaggatcatcaaaatcttatatcaaagcagtaagtatctgtaatgatagatttagattataggttaggagtaggagcattgatagtagcactaatcatagcaatagttgtgtggaccatagtatatatagaatataggaaattggtaagacaaagcaaaataaactggttaattaaaagaattagggaaagagcagaagacagtggcaatgagagtgagggggacactgaggaattatcaacaatggtggatatggggcgtcttaggcttttggatgttaatgatttgtgcggccgcaccaatgacttataagagcgcatttgatctcagcttctttttaaaagaaaaggggggactggaagggttaatttactctaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacatccggagttttacaaagactgctgacacagaagggactttccgctgggactttccactggggcgttccaggaggtgtggtctgggcgggactgggagtggtcaaccctcagatgcggcatataagccgctgcttttcgcttgtactgggtctctctaggtagaccagatctgagcctgggagctctctggctatctagggaacccactgcttaagcctcaataaagcttgccttgagtgctctgagcagtgtgtgcccgtctattgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaaaagcttgcatgcctgcaggtcgactctagaggatcc

SEQ ID NO: 24 - DNA, 3412 bp, Human immunodeficiency virus
gaattcggagtaggagcattgatagtagcactaatcatagcaatagttgtgtggaccatagtatatatagaatataggaaattggtaagacaaagcaaaataaactggttaattaaaagaattagggaaagagcagaagacagtggcaatgagagtgagggggacactgaggaattatcaacaatggtggatatggggcgtcttaggcttttggatgttaatgatttgtaatgggggaggaaacttgtgggtcacagtctattatggggtacctgtgtggaaagaagcaaaaaccactctactctgtgcatcagatgccaaagcatatgagagggaagtgcataatgtctgggctacacatgcctgtgtacccacagaccccaacccacaagaaatagttttgggaaatgtaacagaaaattttaacatgtggaaaaatgacatggtggatcagatgcatgaggatgtaatcagtttatgggatcaaagcctaaagccatgtgtaaaattgaccccactctgtgtcactttagaatgtagaaatgttagcagaaatgttagcagttataatacctacaatgggagcgtggaggaaataaaaaattgctctttcaatgcaaccccagaagtaagagataggaagcagagaatgtatgctctcttttatggacttgatatagtaccacttaataagaagaactctagtgagaactccagtgagtatagattaataaattgtaatacctcagccataacacaagcctgtccaaaggtcacttttgatccaattcctatacactattgtgctccggctggttatgcgattctaaagtgtaataataagacattcaatgggacaggaccatgcaataatgttagtacagtacaatgtacacatggaattaagccagtagtatcaactcaactactgttaaatggtagcctagcagaaggagagataataattagatctgaaaatctgacaaacaatgtcaaaacaataatagtacatcttaatcaatctgtagaaattgtgtgtacaagacccaataataatacaagaaaaagtataaggataggaccaggacaaacattctatgcaacaggagacataataggagacataagacaagcacattgtaacattagtagagataaatggaatgaaactttacaaagggtaggtaaaaaattagcagaacacttccataataagacaataaaatttgcatcatcctcaggaggggacctagaaattacaacacatagctttaattgtagaggagaatttttctattgtaatacatcaggcctgtttaatggtacatacatgcctacatacatgcctaatggtacagaaagtaattcaaactcaactatcacaatcccatgcagaataaagcaaattataaacatgtggcaggaggtaggacgagcaatgtatgcccctcccattgcaggaaacataacatgtacatcaaatatcacaggactactattggtacatgatggaggaataaaggaaaatgatacagagaataagacagagatatttagacctggaggaggagatatgagggacaattggagaagtgaattatataaatataaagtggtagaaattaagccattgggagtagcacccactgcagcaaaaaggagagtggtggagagagaaaaaagagcagtgggaataggagctgtgttccttgggttcttgggagcagcaggaagcactatgggcgcggcgtcaataacgctgacggcacaggccagacaattgttgtctggtatagtgcaacagcaaagcaatttgctgagggctatagaggcgcaacagcatctgttgcaactcacagtctggggcattaagcagctccagacaagagtcctggctatagagagatacctaaaggatcaacagctcctagggatttggggctgctctggaaaactcatctgcactactgctgtaccttggaactccagttggagtaacaaaactcaaagtgagatttggaataacatgacctggatgcagtgggatagagaagttagtaattacacaaacataatatacagcttgcttgaagaatcgcaaaaccagcaggaaaaaaatgaaaaagatttattagcattggacagttggaaaaatctatggagttggtttgacataacaaattggctgtggtatataaaaatattcataatgatagtaggaggcttgataggtttaagaataatttttgctgtgctctctatagtgaatagagttaggcagggatactcacctttgtcgtttcagacccttaccccgaacccaaggggacccgacaggctcggaagaatcgaagaagaaggtggagagcaagacaaagacagatccattcgattagtgaacggattcttagcacttgcctgggacgatctacggaacctgtgcctcttcagctaccaccgattgagagacttcatatcggtggcagcgagagtggtggaacttctgggacgcagcagttgggaagcccttaaatatctgggaagtcttgtgcagtattggggtctggagctaaaaaagagtgctattagtctgtttgatagcatagcaatagtagtagctgaaggaacagataggattatagaattagtacaaggattttgtagagctatccgcaacatacctacaagaataagacagggctttgaagcagctttgcaataaaatggggggcaagtggtcaaaatgcagcatagtaggatggcctgctataagagagagaatgagacgagctgagccagcagcagaaggagtaggagcagcgtctcaagacttagataaacatggagcacttacaagcagcaacacagacaccactaatgctgattgtgcttggctgagagcacaggaggaggaaggagaagtaggctttccagtcacacctcaggtgcctttaagaccaatgacttataagagcgcatttgatctcagcttctttttaaaagaaaaggggggactggaagggttaatttactctaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacatccggagttttacaaagactgctgacacagaagggactttccgctgggactttccactggggcgttccaggaggtgtggtctggggatcc

SEQ ID NO: 25 - DNA, 4217 bp, Human immunodeficiency virus
ctcactatagggcgaattcgagctcggtacccgggaggctgtcagacactttcctagaccatggctccatagcttaggacaacatatttatgaaacctatggggatacttggacaggagttgaagttataataagaatactgcaacaactactgtttattcatttcagaattgggtgccatcatagcagaataggcattattcgacagagaagagcaagaaatggagccaatagatcctaacctagagccctggaatcatccaggaagtcggcctaaaactgcttgcaatcaatgttactgtaaaagatgtagctatcattgtctagtttgctttcagaaaaaaggcttaggcatttcctatggcaggaagaagcggagacagcgacgaagcgctcctccaagcagtgaggatcatcaagatcttatatcaaagcagtaagtatatgtaatgttaactttactagcaagagtagattatagattaggagtaggagcattgatagtagcattaatcatagcaatagtagtgtggatcatagcatatctagaatataggaaattaaaaagacaaataaaaatagacaggttaattagaaaaattagggaaagagcagaagacagtggcaatgagagtgatggggatattgaagaattatcaacaatggtggatatggggcatcttaggcttttggatgctaatgatatataatgtgaggggcttgtgggtcacagtctactatggggtacctgtgtggaaagaagcaaaaactactctattctgtgcatcagatgctaaagcatatgagaaagaagtgcataatgtctgggctacacatgcctgtgtgcccacagaccccaacccacaagaaatggatttgaaaaatgtaacagaaaattttaacatgtgggaaaatgacatggtggatcagatgcatgaggatataatcagtttatgggatgaaagcctaaagccatgtgtaaagttgaccccactctgtgtcactttaaactgtacaaatgctaatgttactgctagtaatggtagcactacctacaataatggcataattggagaaatgaaaaattgctctttcaaggtaactacagaaatgagagataagacgaagaaagaaatggcacttttttataaacttgatatagtaccaattaatagaaaaaactctagaaagaactctagtgagtatagagagtatagattaatacattgtaatacctcaaccataagacaagcctgtccaaaggtctcttttgacccaattcctatacattattgcgctccagctggttttgcgattctaaagtgtaataataagacattcaatggaacagggccatgcaacaaggtcagcacagtacaatgtacacatggaattaagccagtggtatcaactcagctactgttaaatggtagcctagcagaaggagagataataattagatctaaaaatctaacagacaatgccaagacaataatagtacatcttaatgaatctgtagagattgtgtgtataaggcccggcaataatacaaggaaaagggtgagattaggaatagggccaggacaaacattttatgcaacaggacgagtaataagggacataagacaagcacattgtaacattagtgcaaatgcatggaacaaaactttacaaaaggtaggtaaaaaattaaaagaacacttcaataaaataatagtgtttcaaccacattcaggaggagatccagaaattacaacacatagctttaattgtggaggagaatttttctattgcaatacatcaggcctatttaatggtacatttaatggtacatacatgtctaatggtacagagaataattcaaacatcacactccaatgtagaataaaacaaattataaaaatgtggcagggggtaggacaagcaatgtatgcccctcccattcaaggaaacataacatgtaaatcaaatatcacaggactgctattgacacgtgatggaggaacagaaaacggaacagagagaaataatgagacatttagacctggaggaggagatatgaaggacaattggagaagtgaattatacaaatataaagtggtagaaattaagccattaggaatagcacccactagtgcaaaaaggagagtggtggaaagagagaaaagagcagtgggaataggagctgtgttccttgggttcttgggagcagcaggaagcactatgggcgcggcgtcaatagcgctgacggtacaggccagacaattattgtctggtatagtgcaacaacaaagcaatttgctgagggctatagaggcgcaacagcatttgttgcaactcacggtctggggcgttaaacagctccaggcaagagtcctggctctagaaagatacctaacggatcaacagctcctagggatgtggggctgctctggaaaactcatctgcaccactgctgtgccttggaactctagttggaataatagaactgaaagtgagatttggaataacatgacctggatgcagtgggataaagaaattaataattacacagaaacaatatacagattgcttgaagactcgcaaaaccagcaggaacaaaatgaaaaagatttactagcattggacaggtggaacaatctgtggagttggtttagcataacaaattggctatggtatataaaaatattcataatgatagtaggaggcttgatagggttaaggataatttttgctgtgctttctatagtgaatagagttaggcagggatactcacctttgtcatttcagacccttatcccaaacccgaggggactcgacaggctcggaagaatcgaagaagaaggtggagagcaagacagaaacagatccattcgcttagtgaacggattcttagcacttgcctgggacgacctgcggagcctgtgccttttcagctaccaccaattgagagacttcatattaattgtagtgagagcagtggaacttctgggacgcagcagtctcaagggactgcagagggggtgggaagcccttaagtatctgggaagtcttgtgcaatattggggtttggagctaaaaaagagtgctgttagtctgtttgattgcatagcaatagcagtagccacaggaacagataggattatagaagtattacaaggaattggtagagctatcatcaacatacctagaagaataagacagggccttgaagcagctttgcaataaaatggggggcaagtggtcaaaaagcagtatagttggatggcctgctgtaagggaaagaataagacgaactaggccaaatggaagagaaagaataaggcaaactgagccagcagcagaaggagtaggagcagcatctcaagacttagataaatatggagcacttaccagcagcaatacacctggcaataatgctgattgtgcctggctgcaagcgcaggaggaggaagaggacgtaggctttccagtcagacctcaagtgcctttaagaccaatgacatataaggcagcagtagatctcagtcactttttaaaagaaaaggggggactggaagggttaatttactccaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacatccggagttttacaaagactgctgacacagaagggactttccgctgggactttccactggggcgttccaggaggtgtggtctgggcgggactgggagtggtcaaccctcagatgcggcatataagccgctgcttttcgcttgtactgggtctctctaggtagaccagatctgagcctgggagctctctggctatctagggaacccactgcttaagcctcaataaagcttgccttgagtgctctgagcagtgtgtgcccgtctattgtgtgactctggtaactagagatccctcagacccttttggtagtgtggaaaatctctagcatcggtacccggggatcctctagagtcgacc

SEQ ID NO: 26 - DNA, 11035 bp, Human immunodeficiency virus
aattctgtggtttcatttactctaaacctgtgattcctctgaattattttcattttaaagaaattgtatttgttaaatatgtactacaaacttagtagttggaagggttaatttactccaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacacccggagttttacaaagactgctgacacagaagggactttccgctgggactttccactggggcgttccaggaggtgtggtctgggcgggactgggagtggtcaaccctcagatgcggcatataagccgctgcttttcgcttgtactgggtctctctaggtagaccagatctgagcctgggagctctctggctatctagggaacccactgcttaagcctcaataaagcttgccttgagtgctctgagcagtgtgtgcccgtctattgtgtgactctggtaactagagatccctcagacccttttggtagtgtggaaaatctctagcagtggcgcccgaacagggacttgaaagcgaaagtaagaccagagaagatcctctagacgcaggactcggcttgctgaagtgcactcggcaagaggcgagagcggcgactggtgagtacgccaattttatttgactagcggaggctagaaggagagagatgggtgcgagagcgtcaatattaagagggggaaaattagataaatgggaaagaattaggttaaggccagggggaaagaaacactatatgctaaaacacctagtatgggcaagcagggagctggaaagattcgcactcaaccctggccttttagagacagcagaaggctgtaaacaaataataaaacagctacaaccagctcttcagacaggaacagaggaacttaaatcattacacaacacagtagcaactctctattgtgtacatgcagggatagaagtacgagacaccaaagaagccttagacaagatagaggaagaacaaaacaaaattcagcaaaaaacacaacaggcaaaagaggctgacgggaaggtcagtcaaaattatcctatagtgcagaatctccaagggcaaatggtacaccaggccatatcacctagaactttgaatgcatgggtaaaagtaatagaggagaaggcttttagcccagaggtaatacccatgtttacagcattatcagaaggagccaccccacaagacttaaacaccatgttaaatacagtggggggacatcaagcagccatgcaaatgttaaaagataccatcaatgaagaggctgcagaatgggatagattacatccaatccatgcagggcctattgcaccaggccaaatgagagaaccaaggggaagtgacatagcaggaactactagtagccttcaggaacaaatagcatggatgacagggaacccacctgttccagtgggagacatctataaaagatggataattctggggttaaataaaatagtaagaatgtatagccctgttagcattttggacataagacaagggccaaaggaaccctttagagactatgtagaccggttctttaaaactttaagagctgaacaagctacacaagatgtaaaaaattggatgacagacaccttgttggtccaaaatgcgaatccagattgtaagaccattttaagagcattaggaccaggggcttcattagaagagatgatgacagcatgtcagggagtgggaggacctggccacaaagcaagagtgttggctgaggcaatgagccaagcaaacagtaccatactgatgcagagaagcaattttaaaggctctaaaagaattgttaaatgtttcaactgtggcaaggaggggcacatagccaaaaattgcagggcccctaggaaaaaaggctgttggaaatgtggaaaggaaggtcaccgcgtgcgatatcgagcccggtcacctggattcctgagtgggaatttgttaatacccctcccctagtaaaattatggtaccagctggagaaagatcccatagcaggagtagaaactttctatgtagatggagcagctaatagggaaactaagttaggaaaagcagggtatgttactgacagaggaaggcagaaaattgtttctctaactgaaaccacaaatcagaagactgagttgcaagcaatttatctagctttgcaagattcaggatcagaagtaaacatagtaacagattcacagtatgcattagggatcattcaagcacaaccagataagagtgaatcagagttagttaaccaaataatcgaacagttaataaaaaaggaaagggtctatctgtcatgggtaccagcacataaaggaattggaggaaatgaacaagtagataaattagtaagtagtggaatcaggaaagtgctatttctagatggaatagataaagctcaagaagagcatgaaaagtatcacagcaattggagagcaatggccagtgactttaatctaccacccgtagtagcaaaagaaatagtagctagctgtgatcaatgtcagctaaaaggggaagccatgcatggacaagtagactgtagtccagggatatggcaattagattgtacacatttagaaggaaaaatcatcctggtagcagtccatgtagccagtggctacatagaagcagaggttattccagcagaaacaggacaagaaacagcatactttatactaaaattagcaggaagatggccagtcaaagtaatacatacagacaatggtagtaatttcaccagtgctgcagtcaaggcagcctgttggtgggcaggtatccaacaggaatttgggattccctacaatccccaaagtcagggagtagtagaatctatgaataaagaattaaagaaaattatagggcaggtaagagatcaagctgagcaccttaagacagcagtacaaatggcagtattcattcacaattttaaaagaaaaggggggattggggggtacagtgcaggggaaagaataatagacataatagcaacagacatacaaactaaagaattacaaaaacaaattataaaaattcaaaattttcgggtttattacagagacagcagagaccccatttggaaaggaccagccaaactactctggaaaggtgaaggggcagtagtaatacaagataatagtgacataaaggtagtaccacggaggaaagcaaaaatcattaaggactatggaaaacagatggcaggtgctgattgtgtggcaggtagacaggatgaagattagaacatggaatagtttagtaaaacaccatatgtatgtttcaaggagagctaaaggatggttttacagacatcattatgacagcagacatccaaaagtaagttcagaagtacacatcccattaggggaggctagattagtaataaaaacatattgggggttgcaaacaggagaaagagactggcatttgggtcatggagtctccatagaatggagattgagaagatataacacacaaatagaacctggcctggcagaccagctaatccatatgcattattttgattgttttgcagactctgccataaggaaagccatattaggacacatagttattcctaggtgtgactatcaagcaggacataataaggtaggatctctacaatacctggcactgacagcactgataaaaccaaaaaagataaagccacctctgcctagtattaagaaattagtagaggatagatggaacaatccccagaagatcaggggccgcagagggaaccatacaatgaatggacactagagcttctagaggaactcaagcaggaagctgtcagacactttcctagaccatggctccatagcttaggacaacatatttatgaaacctatggggatacttggacaggagttgaagttataataagaatactgcaacaactactgtttattcatttcagaattgggtgccatcatagcagaataggcattattcgacagagaagagcaagaaatggagccaatagatcctaacctagagccctggaatcatccaggaagtcggcctaaaactgcttgcaatcaatgttactgtaaaagatgtagctatcattgtctagtttgctttcagaaaaaaggcttaggcatttcctatggcaggaagaagcggagacagcgacgaagcgctcctccaagcagtgaggatcatcaagatcttatatcaaagcagtaagtatatgtaatgttaactttactagcaagagtagattatagattaggagtaggagcattgatagtagcattaatcatagcaatagtagtgtggatcatagcatatctagaatataggaaattaaaaagacaaataaaaatagacaggttaattagaaaaattagggaaagagcagaagacagtggcaatgagagtgatggggatattgaagaattatcaacaatggtggatatggggcatcttaggcttttggatgctaatgatatataatgtgaggggcttgtgggtcacagtctactatggggtacctgtgtggaaagaagcaaaaactactctattctgtgcatcagatgctaaagcatatgagaaagaagtgcataatgtctgggctacacatgcctgtgtgcccacagaccccaacccacaagaaatggatttgaaaaatgtaacagaaaattttaacatgtgggaaaatgacatggtggatcagatgcatgaggatataatcagtttatgggatgaaagcctaaagccatgtgtaaagttgaccccactctgtgtcactttaaactgtacaaatgctaatgttactgctagtaatggtagcactacctacaataatggcataattggagaaatgaaaaattgctctttcaaggtaactacagaaatgagagataagacgaagaaagaaatggcacttttttataaacttgatatagtaccaattaatagaaaaaactctagaaagaactctagtgagtatagagagtatagattaatacattgtaatacctcaaccataagacaagcctgtccaaaggtctcttttgacccaattcctatacattattgcgctccagctggttttgcgattctaaagtgtaataataagacattcaatggaacagggccatgcaacaaggtcagcacagtacaatgtacacatggaattaagccagtggtatcaactcagctactgttaaatggtagcctagcagaaggagagataataattagatctaaaaatctaacagacaatgccaagacaataatagtacatcttaatgaatctgtagagattgtgtgtataaggcccggcaataatacaaggaaaagggtgagattaggaatagggccaggacaaacattttatgcaacaggacgagtaataagggacataagacaagcacattgtaacattagtgcaaatgcatggaacaaaactttacaaaaggtaggtaaaaaattaaaagaacacttcaataaaataatagtgtttcaaccacattcaggaggagatccagaaattacaacacatagctttaattgtggaggagaatttttctattgcaatacatcaggcctatttaatggtacatttaatggtacatacatgtctaatggtacagagaataattcaaacatcacactccaatgtagaataaaacaaattataaaaatgtggcagggggtaggacaagcaatgtatgcccctcccattcaaggaaacataacatgtaaatcaaatatcacaggactgctattgacacgtgatggaggaacagaaaacggaacagagagaaataatgagacatttagacctggaggaggagatatgaaggacaattggagaagtgaattatacaaatataaagtggtagaaattaagccattaggaatagcacccactagtgcaaaaaggagagtggtggaaagagagaaaagagcagtgggaataggagctgtgttccttgggttcttgggagcagcaggaagcactatgggcgcggcgtcaatagcgctgacggtacaggccagacaattattgtctggtatagtgcaacaacaaagcaatttgctgagggctatagaggcgcaacagcatttgttgcaactcacggtctggggcgttaaacagctccaggcaagagtcctggctctagaaagatacctaacggatcaacagctcctagggatgtggggctgctctggaaaactcatctgcaccactgctgtgccttggaactctagttggaataatagaactgaaagtgagatttggaataacatgacctggatgcagtgggataaagaaattaataattacacagaaacaatatacagattgcttgaagactcgcaaaaccagcaggaacaaaatgaaaaagatttactagcattggacaggtggaacaatctgtggagttggtttagcataacaaattggctatggtatataaaaatattcataatgatagtaggaggcttgatagggttaaggataatttttgctgtgctttctatagtgaatagagttaggcagggatactcacctttgtcatttcagacccttatcccaaacccgaggggactcgacaggctcggaagaatcgaagaagaaggtggagagcaagacagaaacagatccattcgcttagtgaacggattcttagcacttgcctgggacgacctgcggagcctgtgccttttcagctaccaccaattgagagacttcatattaattgtagtgagagcagtggaacttctgggacgcagcagtctcaagggactgcagagggggtgggaagcccttaagtatctgggaagtcttgtgcaatattggggtttggagctaaaaaagagtgctgttagtctgtttgattgcatagcaatagcagtagccacaggaacagataggattatagaagtattacaaggaattggtagagctatcatcaacatacctagaagaataagacagggccttgaagcagctttgcaataaaatggggggcaagtggtcaaaaagcagtatagttggatggcctgctgtaagggaaagaataagacgaactaggccaaatggaagagaaagaataaggcaaactgagccagcagcagaaggagtaggagcagcatctcaagacttagataaatatggagcacttaccagcagcaatacacctggcaataatgctgattgtgcctggctgcaagcgcaggaggaggaagaggacgtaggctttccagtcagacctcaagtgcctttaagaccaatgacatataaggcagcagtagatctcagtcactttttaaaagaaaaggggggactggaagggttaatttactccaagaaaaggcaagaaatccttgatttgtgggtctatcacacacaaggcttcttccctgattggcaaaactacacaccgggaccaggagtcagatacccactgacttttgggtggtgcttcaagctggtaccagttgacccaagggaagtagaagaggccaacgaaggagaagacaactgtttgctacaccctgtgtgccagcatggaatggaggatgaacacagagaagtattaaagtggaagtttgacagtcagctagcacgcagacacatggcccgcgagctacatccggagttttacaaagactgctgacacagaagggactttccgctgggactttccactggggcgttccaggaggtgtggtctgggcgggactgggagtggtcaaccctcagatgcggcatataagccgctgcttttcgcttgtactgggtctctctaggtagaccagatctgagcctgggagctctctggctatctagggaacccactgcttaagcctcaataaagcttgccttgagtgctctgagcagtgtgtgcccgtctattgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaaaagcttgcatgcctgcaggtcgactctagaggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttcgataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcg

SEQ ID NO: 27 - DNA, 17 bp, Human immunodeficiency virus
gtggaaaatctctagca

SEQ ID NO: 28 - DNA, 12 bp, Human immunodeficiency virus
acagggaaccca

SEQ ID NO: 29 - DNA, 25 bp, Human immunodeficiency virus
gtcaccgcgtgcgatatcgagcccg

(19) United States
(12) Patent Application Publication
(10) Pub. No.: US 2012/0064514 A1
(43) Pub. Date: Mar. 15, 2012

(54) HIV-1-C RESISTANCE MONITORING

(75) Inventors: David Nauwelaers, Kortrijk-Dutsel (BE); Lieven Jozef Stuyver, Herzele (BE)

(73) Assignee: Virco BVBA, Mechelen (BE)

(21) Appl. No.: 13/266,973

(22) PCT Filed: May 11, 2010
(86) PCT No.: PCT/EP2010/056450

§ 371 Date: Oct. 28, 2011

(30) Foreign Application Priority Data
May 12, 2009 (EP) 09160003.1

(51) Int. Cl. C12Q 1/70; C12N 15/63

(52) U.S. Cl. 435/5; 435/320.1

(57) ABSTRACT

The present invention relates to methods for the evaluation of HIV-1 Subtype C (HIV-1-C) treatment. The methods are based on evaluating molecular events at the HIV-1-C gag-protease-reverse transcriptase (GPRT) resulting in altered therapeutic efficacy of investigated anti-retroviral compounds. The methods rely on providing HIV-1-C GPRT RNA and evaluating a treatment either through genotyping or phenotyping methods. Said methods may find a use in the field of diagnostics, drug screening, pharmacogenetics and drug development.

This application is the National Stage of PCT Application No. PCT/EP2010/056450 filed May 11, 2010, which claims priority from European Patent Application No. 09160003.1 filed May 12, 2009, the entire disclosures of which are hereby incorporated in their entirety.

The present invention relates to methods for the evaluation of human immunodeficiency virus type I (HIV-1) subtype-C (HIV-1-C) treatment. The methods are based on evaluating molecular events at the HIV-1-C gag-protease-reverse transcriptase (GPRT) coding region, resulting in altered therapeutic efficacy of investigated anti-retroviral compounds. The methods rely on providing HIV-1-C GPRT RNA and evaluating a treatment either through genotyping or phenotyping methods. Said methods may find a use in the field of diagnostics, drug screening, pharmacogenetics and drug development.

There are two types of HIV: HIV-1 and HIV-2. Both types are transmitted by sexual contact, through blood, and from mother to child, and they appear to cause clinically indistinguishable AIDS. However, it seems that HIV-2 is less easily transmitted, and the period between initial infection and illness is longer in the case of HIV-2. Worldwide, the predominant virus is HIV-1, and generally when people refer to HIV without specifying the type of virus they will be referring to HIV-1. The relatively uncommon HIV-2 type is concentrated in West Africa and is rarely found elsewhere. The strains of HIV-1 can be classified into three groups: the “major” group M, the “outlier” group 0 and the “new” group N. Group O appears to be restricted to west-central Africa and group N—discovered in 1998 in Cameroon—is extremely rare. More than 90% of HIV-1 infections belong to HIV-1, group M.

Within group M there are known to be at least nine genetically distinct subtypes (or clades) of HIV-1. These are subtypes A, B, C, D, F, G, H, J and K. The HIV-1 subtypes are very unevenly distributed throughout the world, with the most widespread being subtypes A and C. Subtype C predominates in West and Central Africa (>50% world-wide), while subtype A is possibly responsible for much of the Russian epidemic.

Historically, subtype B has been the most common subtype in Europe, the Americas, Japan and Australia. Although this remains the case, other subtypes are becoming more frequent and now account for at least 25% of new infections in Europe. Subtype D is generally limited to East and Central Africa. Subtype F has been found in Central Africa, South America and Eastern Europe. Subtype G has been observed in West and East Africa and Central Europe. Subtype H has only been found in Central Africa; J only in Central America; and K only in the Democratic Republic of Congo and Cameroon.

The epidemic spread of HIV in sub-Saharan Africa began in the late 1970s and, during the late 1980s, gradually spread to the South of the continent. HIV-1-C was first discovered in North East Africa in the early 1980s and has since also moved to the southern parts of Africa. In addition, the subtype C epidemic has also spread to East and Central Africa where it is becoming the predominant subtype. The C epidemic has also spread to South and Central China, India, Nepal and Brazil. The variants circulating in China are mainly B/C recombinants where the subtype C component appears to have been introduced into China from India.

The subtype C epidemic has now become the most predominant subtype in Southern African countries where HIV prevalence is the highest in the world. Extrapolating the subtype C sequence frequency from the Los Alamos sequence database shows that subtype C accounts for >46% of all infections in sub-Saharan Africa and contributes to just over 50% of global infections. Of all the new infections that occurred globally during the period from 1999 to 2002, 45% are estimated to be subtype C infections, based on the subtype C sequence frequency obtained from published data. It is important to note that this estimate could be greater than the actual prevalence as a result of sequencing bias. However, there is still much evidence suggesting that subtype C is increasing and that there is a definite need to monitor and describe the viruses circulating in countries with high subtype C infections.

A number of different therapeutic regimens have been developed to treat HIV infection, mainly subtype B. However, like many viruses, HIV has no proofreading capacity; thus, it can quickly mutate to overcome the effects of new drugs targeted against it. Under the selective pressure of a given therapy, the virus mutates to phenotypes that reduce or eliminate the effects of the administered drugs. Despite the development of new classes of anti-HIV drugs such as protease (PR) and reverse transcriptase (RT) inhibitors, drug resistance continues to increase. Further, drug-resistant virus strains can infect new individuals, gradually replacing the more treatable strains in the infected population.

The ease with which HIV can mutate under the selective pressure of drug therapy requires the frequent monitoring of the replicative capacity of a patient's virus in response to the patient's current therapy so that the therapeutic strategy can be adjusted or changed to provide maximum benefit over time. Often, the physician must change the doses of drugs, or initiate combination therapy using protease and reverse transcriptase inhibitors, or other types of anti-HIV drugs.

Accurate determination of the susceptibility of a patient's virus strain toward a variety of drugs or drug combinations is especially helpful in making decisions about appropriate treatment. In order to reduce drug resistance and assist physicians in choosing the best therapy for a given HIV-infected patient, sophisticated patient monitoring techniques have been developed, such as Antivirogram™ (described in WO 97/27480 and U.S. Pat. No. 6,221,578 B1). This assay determines the resistance of patient borne virus towards a defined drug regimen by providing information about the susceptibility of the patient's virus strain to protease and reverse transcriptase inhibitor treatment.

The Antivirogram™ assay determines the phenotype of a patient's pol genes. These coding regions are obtained from patient samples, reverse transcribed and amplified by the polymerase chain reaction (PCR), then inserted into a plasmid to create chimeric viruses. The ability of these viruses to invade and kill cells in culture is assessed in the presence of HIV reverse transcriptase and protease inhibitors.

Thus obtained phenotypic and genotypic data enable the development of a database comprising both phenotypic and genotypic information, as described in WO 00/73511. Such a database can further be used to predict the phenotype of a HIV protease or reverse transcriptase gene based on its genotypic profile.

Although most current HIV-1 antiretroviral drugs were designed for use against subtype B, there is no compelling evidence that they are any less effective against other subtypes. Nevertheless, some subtypes may be more likely to develop resistance to certain drugs, have inherent resistance present in their “wild type” sequence or the types of mutations associated with resistance may vary.

Therefore an alternative phenotypic drug resistance assay, which could determine protease and reverse-transcriptase inhibitor-associated resistance of HIV-1 subtype C (HIV-1-C), constitutes a high medical need for health workers and patients to be aware of the subtype they are testing for and of the limitations of the test they are applying. The current invention relates to the construction of a HIV-1-C backbone for use in a recombinant phenotypic drug resistance assay to determine protease and reverse-transcriptase inhibitor-associated resistance and to investigate possible backbone-dependent (subtype B vs. subtype C) resistance profile differences.

One embodiment of the invention relates to an in vitro method for designing a drug regimen for an HIV-1-C infected patient by determining the phenotypic susceptibility of HIV-1-C to at least one drug, comprising:
i) using at least one sample comprising HIV-1 RNA from a patient infected with HIV-1-C, wherein the sample comprises the HIV-1 gag-protease-reverse transcriptase coding region;
ii) reverse-transcribing and amplifying said HIV-1 RNA with primers specific for the HIV-1 gag-protease-reverse transcriptase (GPRT) coding region to obtain at least one amplicon comprising the HIV-1 GPRT coding region, wherein at least one primer is selected from

SEQ ID NO: 1
5′-GCCCCTAGGAAAAAGGGCTGTTGG-3′
(5′ Out)
SEQ ID NO: 2
5′-CATGAGAAATATCACAGTAATTGGAGAGCAATG-3′
(3′ RT)

and nested PCR amplification

SEQ ID NO: 3
5′-AATGTGGAAAGGAAGGACACCAAATGAAAG-3′
(5′ Infusion)
SEQ ID NO: 4
5′-CTCATAACCGTTCGGTGGACCTAAGGACT-3′
(3′ Infusion)

iii) generating a plasmid comprising a reference HIV-1-C sequence with a deletion of the HIV-1 GPRT coding region;
iv) preparing at least one recombinant virus by recombination or ligation between at least one amplicon obtained in step ii) and the plasmid comprising the reference HIV-1-C sequence with a deletion of the HIV-1 GPRT coding region obtained in step iii), and
v) monitoring at least one recombinant virus in the presence of at least one drug to determine the phenotypic susceptibility of HIV-1-C to at least one drug,
   wherein said susceptibility is determined by the cytopathogenicity of said recombinant virus to cells or by determining the replicative capacity or infectivity of said recombinant virus in the presence of at least one drug.

Part of the invention is also a method of constructing a genotypic and phenotypic database of GPRT sequences from HIV-1-C, comprising:
i) obtaining samples of HIV-1-C RNA comprising the GPRT coding region or a portion thereof
ii) reverse-transcribing and amplifying said HIV-1 RNA with primers selected from SEQ ID NO: 1-4 specific for the GPRT coding region of the HIV-1-C genome to obtain an amplicon comprising the GPRT coding region or a portion thereof
iii) determining the nucleotide sequence of the amplicons or portions thereof by using sequencing primers

SEQ ID NO: 5
5′-GAGAGCTTVAGGTTTGGGG-3′
(F1)
SEQ ID NO: 6
5′-AATTGGGCCTGAAAATCC-3′
(F2)
SEQ ID NO: 7
5′-CCTCCATTCCTTTGGATGGG-3′
(F3)
SEQ ID NO: 8
5′-CACTCTTTGGCAACGACCC-3′
(F5)
SEQ ID NO: 9
5′-CTCCCACTCAGGAATCC-3′
(R1)
SEQ ID NO: 10
5′-CTTCCCAGAAGTCTTGAGTTC-3′ (R3)
SEQ ID NO: 11
5′-GGGTCATAATACACTCCATG-3′
(R5)
SEQ ID NO: 12
5′-GGAATATTGCTGGTGATCC-3′
(R6)
SEQ ID NO: 13
5′-CAGACCAGAGCCAACAGCCCC-3′
(F4)
SEQ ID NO: 14
5′-GGTACAGTATTAGTAGGACC-3′
(F6)
SEQ ID NO: 15
5′-GTACTGGATGTGGGTGATGC-3′
(F7)
SEQ ID NO: 16
5′-GTGGGAAAATTGAATTGGG-3′
(F8)
SEQ ID NO: 17
5′-GTACTGTCCATTTATCAGG-3′
(R2)
SEQ ID NO: 18
5′-CTAACTGGTACCATAATTTCACTAAGGGAGG-3′
(R4)
SEQ ID NO: 19
5′-CATTGTTTAACTTTTGGGCC-3′
(R7)
SEQ ID NO: 20
5′-GATAAAACCTCCAATTCC-3′
(R8)

iv) preparing recombinant virus by homologous recombination or ligation between the amplicons and a plasmid comprising the wild-type HIV-1-C sequence with a deletion in the GPRT coding region of the HIV-1-C genome
v) determining the relative replicative capacity of the recombinant virus in the presence of anti-HIV drugs compared to HIV-1-C with a wild-type GPRT coding region sequence
vi) correlating the nucleotide sequence and relative replicative capacity in a data table.

Also a database comprising genotypic and phenotypic data of HIV-1-C GPRT coding regions, wherein the database further provides a correlation between genotypes and between genotypes and phenotypes, wherein the correlation is indicative of efficacy of a given drug regimen is part of the current invention. Such a database can further be used to predict the phenotype of HIV-1-C GPRT coding region based on its genotypic profile.

In the present invention HIV refers to any sample comprising at least one HIV. Since a patient may have HIV in his body with different mutations in the GPRT coding region, it is to be understood that a sample may contain a variety of different HIV containing different mutational profiles in the GPRT coding region. A sample may be obtained for example from an individual, from cell cultures, or generated using recombinant technology, or cloning.

HIV strains compatible with the present invention are any such strains that are capable of infecting mammal cells, particularly human cells. Viral strains used for obtaining a plasmid are preferably clinical HIV-1 sequences, but may also comprise artificial sequences generated by e.g. Synthetic Biology.

Instead of viral RNA, HIV DNA, e.g. proviral DNA, may be used for the methods described herein. In case RNA is used, reverse transcription into DNA by a suitable reverse transcriptase is needed. The protocols describing the analysis of RNA are also amenable for DNA analysis. However, if a protocol starts from DNA, the person skilled in the art knows that no reverse transcription is needed. The primers designed to amplify the RNA strand, also anneal to, and amplify DNA (SEQ ID NO: 1 and 2). Reverse transcription and amplification may be performed with a single set of primers. Suitably a hemi-nested and more suitably a nested approach may also be used to reverse transcribe and amplify the genetic material (SEQ ID NO: 3 and 4). Nucleic acid may be amplified by techniques such as polymerase chain reaction (PCR), nucleic acid sequence based amplification (NASBA), self-sustained sequence replication (3SR), transcription-based amplification (TAS), ligation chain reaction (LCR). Preferably PCR is used.

For the purpose of the present invention an amplicon refers to the amplified and, where necessary, reverse-transcribed GPRT coding region or portions thereof. Additionally, the amplicon may include the flanking regions of the GPRT coding region or portions thereof. It should be understood that this GPRT coding region may be of diverse origin, including plasmids and patient material; suitably it is obtained from patient derived material. A portion of the GPRT coding region is defined as a fragment of GPRT coding region recovered from patient borne virus, lab virus strains including mutant virus strains or artificial HIV DNA sequences.

Primers specific for the GPRT coding region of the HIV genome such as the primers described herein and their homologs are chosen from SEQ. ID N° 1-4 or have at least 80% homology, preferably 90% homology, more preferably 95% homology as determined using algorithms known to the person skilled in the art such as FASTA and BLAST. Interesting sets of primers include at least one primer selected from SEQ. ID N° 1-4 . The primer sequences listed herein may be labelled. Suitably, this label may be detected using fluorescence, luminescence or absorbance. In addition primers located in a region of 50 nucleotides (nt) upstream or downstream from the sequences given herein constitute part of the present invention. Specifically, the primers may be located in a region of 20 nt upstream or downstream from the sequences given herein and, constitute, as well, part of the present invention. Also, primers comprising at least 8 consecutive bases present in either of the primers described herein constitute an embodiment of the invention. In one aspect of the present invention the primers may contain linker regions for cloning. Optionally, the linker region of a primer may contain a restriction enzyme recognition site. Preferably, said restriction enzyme recognition site is a unique restriction enzyme recognition site. Alternatively, primers may partially anneal to the target region.

A drug means any agent such as a chemotherapeutic antiretroviral compound or peptide. Examples of drugs include HIV protease inhibitors including ritonavir, amprenavir, darunavir, nelfinavir; reverse transcriptase inhibitors such as nevirapine, delavirdine, etravirine, rilpivirine, AZT or didanosine.

Treatment or treatment regimen refers to the management or handling of an individual medical condition by the administration of drugs, at directed dosages, time intervals, duration, alone or in different combinations, via different administration routes, in suitable formulations, etc.

The susceptibility of at least one HIV to at least one drug is determined by the replicative capacity of the recombinant virus in the presence of at least one drug relative to the replicative capacity of HIV with a wild-type GPRT coding region sequence in the presence of the same at least one drug. Replicative capacity means the ability of the virus or chimeric construct to (re)infect under culturing conditions. This is sometimes referred to as viral fitness. The culturing conditions may contain triggers that influence the growth of the virus, examples of which are drugs.

An alteration in viral drug sensitivity is defined as a change in susceptibility of a viral strain to said drug. Susceptibilities are generally expressed as ratios of EC₅₀ or EC₉₀ values. The EC₅₀ or EC₉₀ value is the effective drug concentration at which 50% or 90% respectively of the viral population is inhibited from replicating. The IC₅₀ or IC₉₀ value is the drug concentration at which 50% or 90% respectively of the enzyme activity is inhibited. Hence, the susceptibility of a viral strain can be expressed as a fold change in susceptibility, wherein the fold change is derived from the ratio of, for instance, the EC₅₀ or IC₅₀ values of a mutant viral strain, compared to the wild type EC₅₀ or IC₅₀ values. In particular, the susceptibility of a viral strain or population may also be expressed as resistance of a viral strain, wherein the result is indicated as a fold increase in EC₅₀ or IC₅₀ as compared to wild type EC₅₀ or IC₅₀.

The susceptibility of at least one HIV to one drug may be tested by determining the cytopathogenicity of the (recombinant) virus to cells. In the context of this invention, the cytopathogenic effect means, the viability of the cells in culture in the presence of (chimeric) viruses. The cells may be chosen from T cells, monocytes, macrophages, dendritic cells, Langerhans cells, hematopoietic stem cells or, precursor cells, MT4 cells and PM-1 cells. Suitable host cells for homologous recombination of HIV sequences include MT4 and PM-1. MT4 is a CD4⁺ T-cell line containing the CXCR4 co-receptor. The PM-1 cell line expresses both the CXCR4 and CCR5 co-receptors. All the above mentioned cells are capable of producing new infectious virus particles upon recombination or ligation of the GPRT deletion vectors with the GPRT amplicons. Thus, they can also be used for testing the generation and spreading of recombinant viruses. The generation and spreading of recombinant viruses may, for example, be monitored by the presence of a reporter molecule including reporter genes.

A reporter or indicator gene is defined as a gene which product has reporting capabilities.

Suitable reporter molecules include tetrazolium salts, green fluorescent proteins, beta-galactosidase, chloramfenicol transferase, alkaline phosphatase, and luciferase.

The term chimeric means a construct comprising nucleic acid material from different origin such as, for example, a combination of wild type virus with a laboratory virus, a combination of wild type sequence and patient derived sequence.

Any type of patient sample may be used to obtain the GPRT coding region, such as, for example, serum and tissue. Viral RNA may be isolated using known methods such as described for instance in Boom, R. et al. (J. Clin. Microbiol. 28(3): 495-503 (1990). Alternatively, a number of commercial methods such as the MDx extraction robot (QIAamp Virus BioRobot MDx Kit, Qiagen, Inc.) and EasyMAG (BioMérieux) may be used to obtain viral RNA from bodily fluids such as plasma, serum, or cell-free fluids. DNA and/or RNA may be extracted from tissue using methods known by the skilled in the art such as the procedure described by Maniatis et al. (1982) which involves the preparation of a cell lysate followed by digestion with proteinase K, obtaining nucleic acid purification by a multi-step phenol extraction, ethanol precipitation and ribonuclease digestion. Optionally, available commercial methods may also be employed to obtain nucleic acids from bodily fluids, such as QIAAMP® Blood kits for nucleic acids isolation from blood and body fluids (Qiagen, Inc.).

Following the generation of a recombinant construct the chimeric virus may be grown and the viral titer determined (expressed as multiplicity of infection, MOI) before proceeding to the determination of the phenotypic susceptibility. The indicator gene, encoding a signal indicative of replication of the virus in the presence of a drug or indicative of the susceptibility of the virus in the presence of a drug may be present in the culturing cells such as MT-4 cells. Alternatively, said indicator gene may be incorporated in the chimeric construct introduced into the culturing cells or may be introduced separately. Suitable indicator genes encode for fluorescent proteins, particularly green fluorescent protein or mutant thereof. In order to allow homologous recombination or transfer of the complete genome assembled through Infusion reagens (Clontech), genetic material may be introduced into the cells using a variety of techniques known in the art including, calcium phosphate precipitation, liposomes, viral infection, and electroporation (Amaxa). The monitoring may be performed in high throughput.

The protocols and products of the present invention may be used for diverse diagnostic, clinical, toxicological, research and forensic purposes including, drug discovery, designing patient therapy, drug efficacy testing, and patient management. The present methods may be used in combination with other assays. The results may be implemented in computer models and databases.

Results from phenotyping and genotyping experiments can be used to develop a database of replicative capacity levels in the presence of particular drugs, drug regimens or other treatment for a large number of mutant HIV strains. One such approach is virtual phenotyping (WO 01/79540). Briefly, the genotype of a patient derived GPRT sequence may be correlated to the phenotypic susceptibility of said patient derived GPRT sequence. If no phenotyping is performed, the sequence may be screened towards a collection of sequences present in a database. Identical sequences are retrieved and the database is further interrogated to identify if a corresponding phenotype is known for any of the retrieved sequences. In this latter case a virtual phenotype may be determined.

A report may be prepared including the EC₅₀ of the viral strain for one or more therapies, the sequence of the strain under investigation and biological or clinical cut-offs, if appropriate. Suitably, complete sequences will be interrogated in the database. Optionally, portions of sequences, such as combinations of mutations indicative of a change in drug susceptibility, may as well be screened. Such combination of mutations is sometimes referred to as a hot-spot (see e.g. WO 01/79540). Additionally, data may then be incorporated into existing programs that analyze the drug susceptibility of viruses with mutations in other segments of the HIV genome such as in the env genes. For example, such a database may be analyzed in combination with reverse transcriptase and protease sequence information and the results used in the determination of appropriate treatment strategies.

Primers used in the invention are listed in the Sequence Listing having SEQ ID NO 1-20.

SEQ ID NO: 1
5′-GCCCCTAGGAAAAAGGGCTGTTGG-3′
SEQ ID NO: 2
5′-CATGAGAAATATCACAGTAATTGGAGAGCAATG-3′
SEQ ID NO: 3
5′-AATGTGGAAAGGAAGGACACCAAATGAAAG-3′
SEQ ID NO: 4
5′-CTCATAACCGTTCGGTGGACCTAAGGACT-3′
SEQ ID NO: 5
5′-GAGAGCTTCAGGTTTGGGG-3′
SEQ ID NO: 6
5′-AATTGGGCCTGAAAATCC-3′
SEQ ID NO: 7
5′-CCTCCATTCCTTTGGATGGG-3′
SEQ ID NO: 8
5′-CACTCTTTGGCAACGACCC-3′
SEQ ID NO: 9
5′-CTCCCACTCAGGAATCC-3′
SEQ ID NO: 10
5′-CTTCCCAGAAGTCTTGAGTTC-3′
SEQ ID NO: 11
5′-GGGTCATAATACACTCCATG-3′
SEQ ID NO: 12
5′-GGAATATTGCTGGTGATCC-3′
SEQ ID NO: 13
5′-CAGACCAGAGCCAACAGCCCC-3′
SEQ ID NO: 14
5′-GGTACAGTATTAGTAGGACC-3′
SEQ ID NO: 15
5′-GTACTGGATGTGGGTGATGC-3′
SEQ ID NO: 16
5′-GTGGGAAAATTGAATTGGG-3′
SEQ ID NO: 17
5′-GTACTGTCCATTTATCAGG-3′
SEQ ID NO: 18
5′-CTAACTGGTACCATAATTTCACTAAGGGAGG-3′
SEQ ID NO: 19
5′-CATTGTTTAACTTTTGGGCC-3′
SEQ ID NO: 20
5′-GATAAAACCTCCAATTCC-3′

### BRIEF DESCRIPTION OF THE DRAWINGS

FIG. 1 Fragment I (A) and Fragment II (B) were digested using BstEII and EcoRI and religated resulting in an HIV-1 subtype C clone lacking a part of GAG, protease and reverse Transcriptase and most of ENV (Fragment I-II (C)). Fragment I-II was linearized using PacI and AccIII to insert the Env region from Fragment III (D) resulting in a final clone, pGEM-HIV-1-C-Δgprt-BstEII-V, that can be linearized using BstEII/EcoRV, ready for In-Fusion cloning with the 1.7 kb GPRT amplicon. pGEM-HIV-1-C-Δgprt-BstEII+GPRT (wild type sequence)

FIG. 2 Experimental flow of the subtype C GPRT amplicon through the pGEM-HIV-1-C-Δgprt-BstEII-V (pHIV-1-C-Δgprt) backbone and the pGEM-HXB2-Δgprt-BstEII (pHIV-1-B-Δgprt) backbone.

FIG. 3 Scatter plot of linked fold changes between HIV-1 subtype C and HIV-1 Subtype B backbone. Scatter plots of FC in subtype B (X-axis) and subtype C (Y-axis). Black line x=y; (A) all drug classes (R2=0.88); (B) NRTIs (R2=0.88); (C) NNRTIs (R2=0.90); (D) PIs (R2=0.87). Analysis of the pairs wise comparison of differences in FCs per clone and per drug, P-value (Red squares) and Ratio FC Subtype B/FC Subtype C (Black diamonds) (E).

FIG. 4 The effect of RAM 184V on the NRTI FC in a subtype B and C backbone. “B” =HIV-1 subtype B backbone; “C”=HIV-1 subtype C backbone; “-”=mutation 184V is not present in RT; “184V”=mutation 184V is present in RT; “Count”=number of observed FC. P values have been calculated for each subtype for FC with mutation vs. FC without mutation.

### EXAMPLE SECTION

### HIV-1-C Resistance Monitoring

Methods for the evaluation of HIV-1 Subtype C (HIV-1-C) treatment are described hereafter. The methods are based on evaluating molecular events at the HIV-1-C gag-protease-reverse transcriptase (GPRT) coding region, resulting in altered therapeutic efficacy of investigated anti-retroviral compounds. The methods rely on providing HIV-1-C GPRT RNA and evaluating a treatment either through genotyping or phenotyping methods. Said methods may find a use in the field of diagnostics, drug screening, pharmacogenetics and drug development.

An HIV-1-C backbone was synthesized for use in a recombinant virus assay to determine phenotypic protease and reverse transcriptase inhibitor-associated resistance and to investigate possible backbone-dependent (subtype B vs. C) resistance profile differences.

An HIV subtype C backbone was designed in silico. The complete genome (12 721 bp) was divided into 4 fragments which were chemically synthesized and subsequently joined together by traditional subcloning. Gag-protease-reverse-transcriptase (GPRT) fragments from 8 patient samples infected with subtype C HIV-1 were RT-PCR amplified. The 1.7 kb PCR fragment was cloned into the HIV-1-C backbone (deleted for GPRT) using In-Fusion reagents. Full-genome clones (N ranging from 1 to 5 per patient sample) were transfected in MT4-eGFP cells where cyto-pathogenic effect (CPE), p24 and Viral Load (VL) were monitored. The resulting HIV-1-C recombinant virus stocks (RVSs) were added to MT4-eGFP cells in the presence of serial dilutions of antiretroviral drugs (PI, NNRTI, N(t)RTI) to determine the fold-change in IC₅₀ compared to the IC₅₀ of wild-type HIV-1 virus. Additionally, viral RNA was extracted from the HIV-1-C RVSs and submitted to an RT-PCR. The resulting GPRT amplicons were recombined into a subtype B backbone and phenotyped as described above, allowing the comparison of GPRT resistance profiles in the two backbones.

Infection of recombinant viruses generated in the HIV-1-C backbone seemed to spread less fast than viruses generated in the subtype B backbone. Also, no CPE was observed in MT4 cells. High titers could be established after reculturing the RVSs in fresh MT4-eGFP cells, confirmed by VL and p24 measurements. Drug resistance profiles generated in both backbones were very similar, including re-sensitizing effects like M184V on AZT.

An HIV-1 subtype C backbone for a recombinant virus phenotyping assay was developed. The resulting recombinant viruses seemed less virulent (e.g., no CPE) but generated similar resistance profiles compared to the profiles obtained in an HIV-1 subtype B backbone.

### Detailed Experimental Approach

### 1. Design and Construction of the HIV-1 Subtype C Backbone

### 1.1. Initial Sequence Design, Synthesis and Construction of the HIV-1 Subtype C Backbone.

The in silico design of the HIV-1 subtype C backbone was based on the subtype C sequence with accession number AB023804 (www.hiv.lanl.gov). This sequence lacked part of the 3′LTR region, which was completed by adding the matching bases as present in the 5′ LTR (5′-GTGGAAAATCTCTAGCA-3′) (SEQ ID NO 27). A BstEII restriction site present at position 1534 (acaGGTAACCca—coding for Thr-Gly-Asn-Pro in GAG) was changed to “acaGGGAACCca” (SEQ ID NO 28) conserving the translation. Also an AccIII restriction site (TCCGGA) at position 308 (5′ LTR) was modified to CCCGGA for cloning purposes (see below).

### 1.2. Synthetic Production of the HIV-1 Subtype C Backbone.

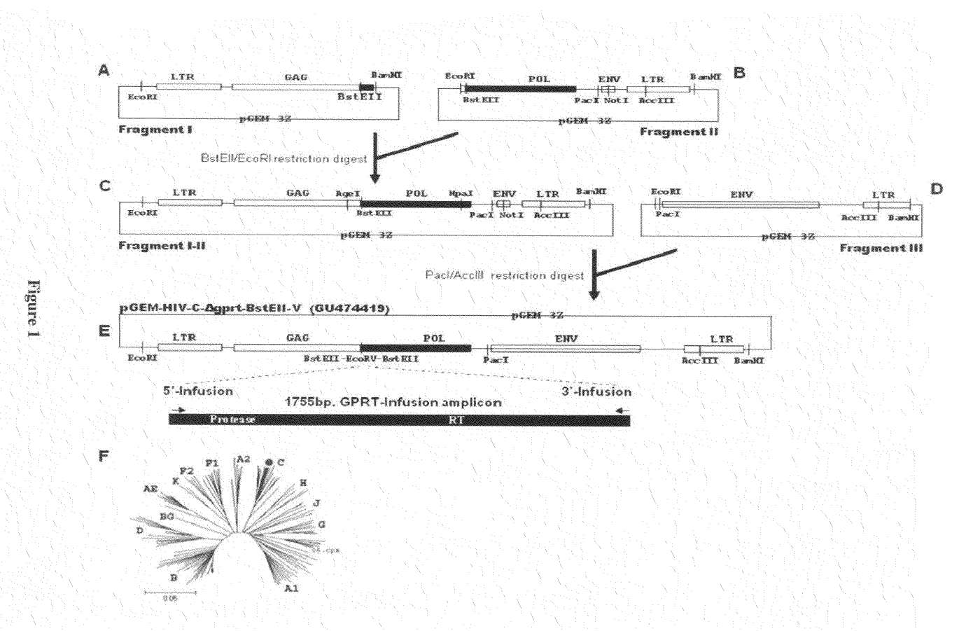

The final design of the subtype C sequence was split in 4 different fragments of which three fragments (flanked by EcoRI and BamHI restriction sites for cloning purposes) were destined for synthesis (FIG. 1, fragments I, II, III). The fragment containing the protease and reverse transcriptase region was not synthesized but PCR-amplified from clinical samples as described above. The synthesis of the 3 DNA fragments was performed as follows: briefly, padded sequences were parsed into contiguous segments of equal length on both the forward and reverse strands. Each segment was then chemically synthesized as an oligonucleotide using GeneWriter™ (Centocor) technology and purified by reversed phase HPLC (Dionex, Sunnyvale, Calif.). The purified oligonucleotides were assembled into the full-length fragments using gene assembly technology (GeneAssembler™, Centocor) and cloned into a pGEM-3z vector (2743 bp) using EcoRI and BamHI (FIG. 1): Vector Fragment-I (FIG. 1-A) (SEQ ID NO 21) contained an EcoRI-BamHI flanking fragment of HIV-1 5′-LTR and GAG, as well as an inserted BstEII restriction site and a small downstream part of POL (2205 bp). Vector Fragment-II (FIG. 1-B) contained an EcoRI-BamHI flanking fragment of HIV-1 GAG (SEQ ID NO 22), as well as an inserted BstEII restriction site, the 3′ part of POL, a fragment of ENV (mostly deleted and replaced with a NotI-containing sequence) and the 3′-LTR (3460bp) (SEQ ID NO 23). While the V3 envelope region of AB023804 was predicted to be R5-tropic according to the Geno2Pheno prediction tool (http://coreceptor.bioinf.mpi-inf.mpg.de/index.php) and Position Specific Scoring Matrices (PSSM, http://indra.mullins.microbiol. washington.edu), (Table I, SEQ ID NO 24) an R4-tropic virus was needed for the transfection assay in MT4 host cells. An envelope sequence retrieved from Los Alamos (subtype C clone C.ZA.01.01ZARP1) was predicted to be X4-tropic (Table I) and was used to design Vector Fragment-III (FIG. 1-D) (SEQ ID NO 25): an EcoRI-BamHI flanking fragment of the complete HIV-1 ENV and the upstream part of the 3′LTR (3412 bp).

### 1.3. Subcloning of the HIV-1 Subtype C Backbone.

In a first step vector Fragment-I and vector Fragment-II were joined by subcloning the EcoRI-BstEII fragment from Vector Fragment-I in Vector Fragment-H digested with the same enzymes. This resulted in an HIV-1 subtype C clone (Vector Fragment-I-II—FIG. 1-C) that had both the majority of POL (replaced by BstEII) and ENV (replaced by NotI) deleted.

The PacI-AccIII fragment of Vector Fragment-III (FIG. 1-D) was subcloned in Vector Fragment-I-II (FIG. 1-C) digested with the same enzymes. This resulted in a Vector Fragment-I-II-III which only had the GPRT region deleted, called “pGEM-HIV-1-C-Δgprt-BstEII”. Finally, the vector was linearized by BstEII and a small artificial sequence (5′-GTCACCGCGTGCGATATCGAGCCCG-3′) (SEQ ID NO 29) was inserted transforming the BstEII site into a BstEII-EcoRV-BstEII site, to reduce the background during In-Fusion and transformation into competent cells. This vector was called “pGEM-HIV-1-C-Δgprt-BstEII-V” (FIG. 1-E) (SEQ ID NO 26). The linearized vector enabled In-Fusion cloning with the 1.7 GPRT-In-Fusion amplicon, restoring a full genome, infectious HIV-1 clone (FIG. 1-E, Genbank reference GU474419). In a phylogenetic tree, the pGEM-HIV-1-C-Δgprt-BstEII-V sequence (completed with a wild-type subtype C GPRT sequence) clustered together with the other HIV-1 subtype C sequences (FIG. 1-F).

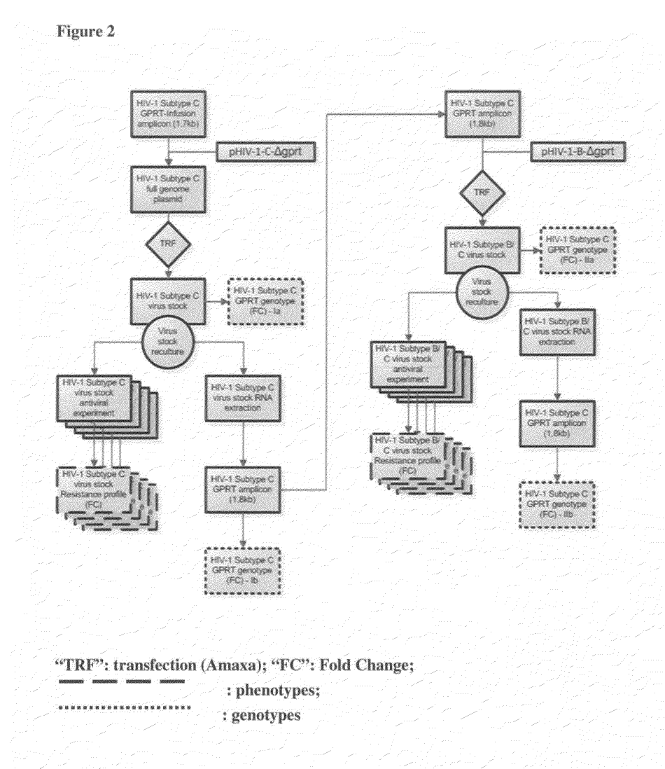

### 2. Generation of Full HIV-1 Genomes (FIG. 2)

### 2.1 HIV-1 Subtype C Backbone

The linearized pGEM-HIV-1-C-Δgprt-BstEII backbone was combined with the purified GPRT-In-Fusion amplicon in a molar ratio 1:7 (final volume of 10 μl) and mixed with the dried reaction beads for In-Fusion according to the guidelines of the manufacturer (In-Fusion™ 2.0 Dry-Down PCR Cloning Kit—Clontech, Cat. No. 639607 (24 rxns), 639608 (96 rxns)), prior to transformation into bacterial cells.

### 2.2 HIV-1 Subtype B Backbone

In contrast to the In-Fusion strategy for the subtype C backbone, a homologous recombination event strategy was used for the subtype B backbone to generate infectious virus. Here the BstEII-linearized pGEM-HXB2Δgprt-BstEII backbone was co-transfected with the 1.8 kb GPRT fragment in an MT4 cell line, resulting in a full-genome infectious virus.

3. Transformation into MAX Efficiency® Stbl2™ Cells.

A total of 10 μl of diluted In-Fusion reaction mix (dilution prepared during In-Fusion cloning—see 2.6.) was added to the MAX Efficiency® Stbl2™ cells (Invitrogen, Cat. No. 10268-019) and treated according to the guidelines of the manufacturer. LB ampicillin agar plates were incubated at 30° C. for approximately 24 hours.

### 4. Phenotyping Assay for Envelope (ENV)

Alternatively, (Vector Fragment-I-II—FIG. 1-C) may be cleaved with AgeI and HpaI to remove the partial Gag-Pol fragment containing the BstEII site to insert the fragment from vector IV (SEQ ID NO 22) cleaved with the same restriction enzymes to obtain a “pGEM-HIV-1-C-Δenv-NotI” vector. This NotI-linearized vector may be used in combination with amplicons spanning the envelope region of HIV to generate complete HIV genomes differentiating in the envelope sequence. These complete HIV genomes may be used to determine the tropism of HIV-1-C viruses when transfected in e.g. GHOST cells bearing either the CXCR4 or CCR5 co-receptor.

The experimental approach can be found in FIG. 2.

5. Antiviral Drug Susceptibility Testing of Virus Stocks Generated with the pGEM-HIV-1-C-Δgprt-BstEII vs. the pGEM-HXB2-Δgprt-BstEII Backbone but Carrying Identical GPRT Fragments.

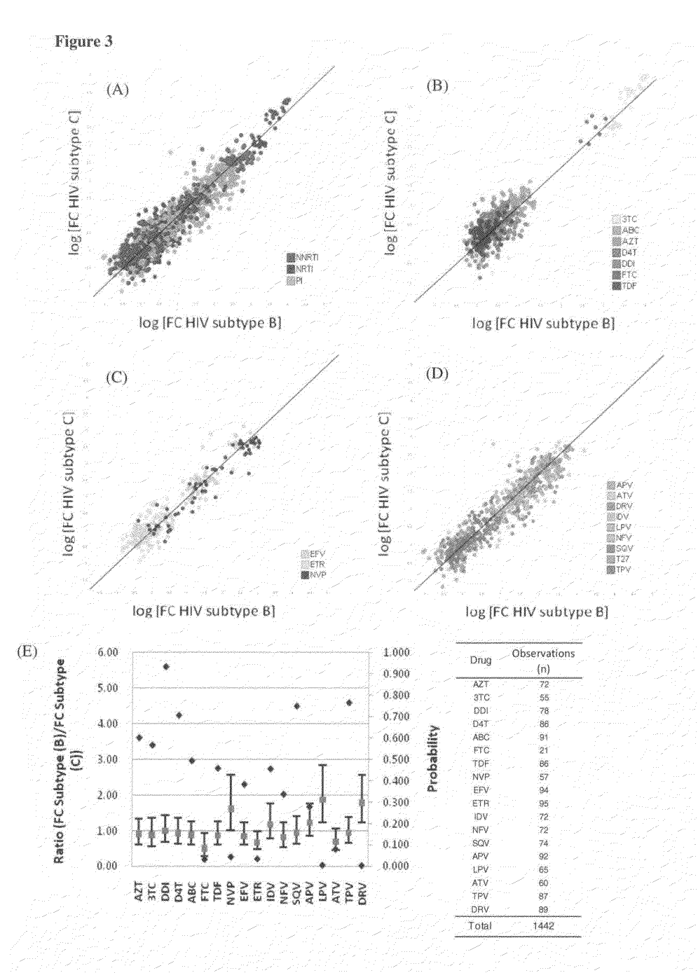

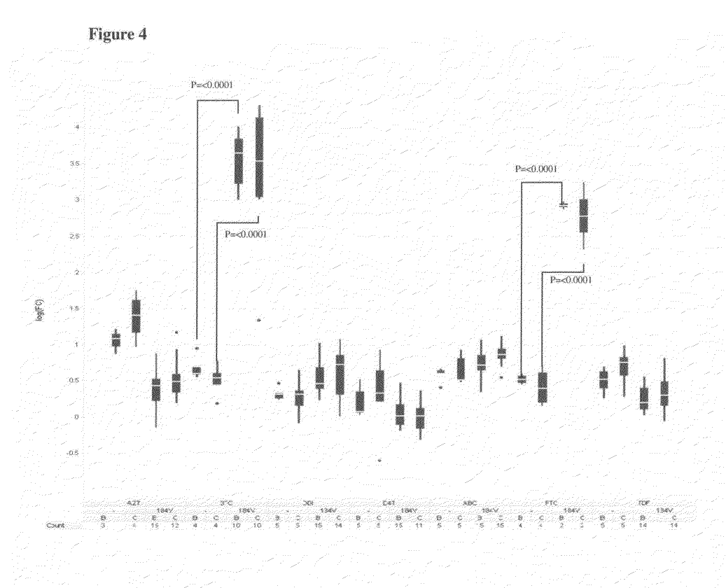

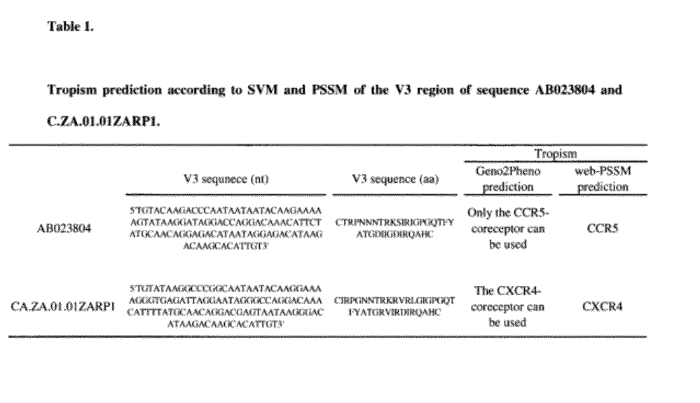

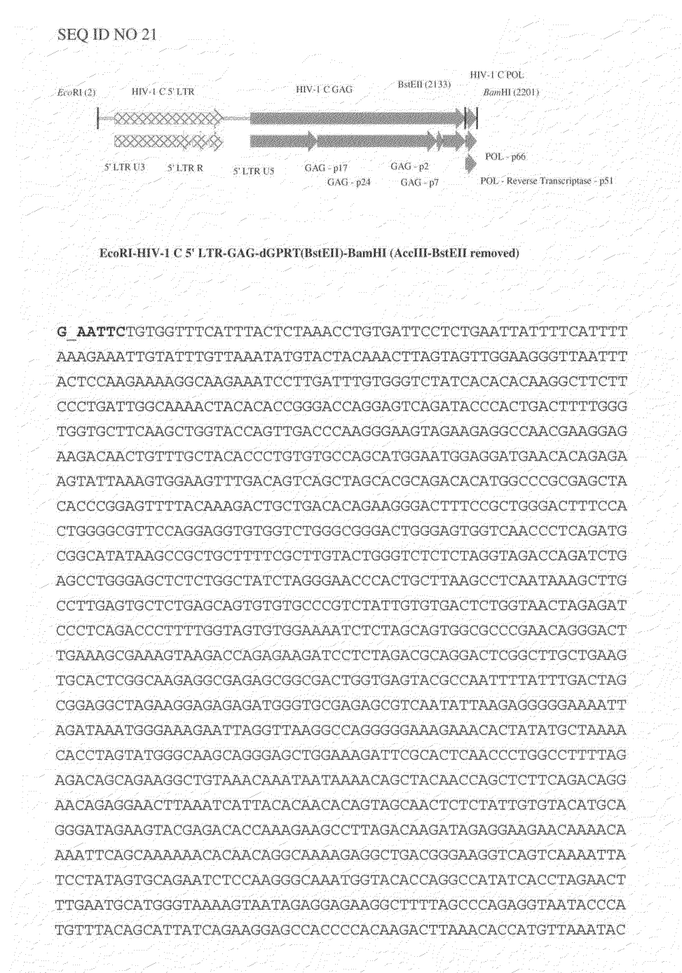

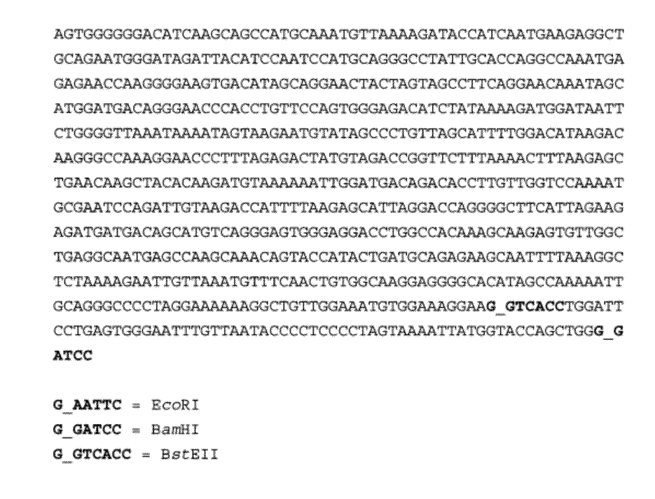

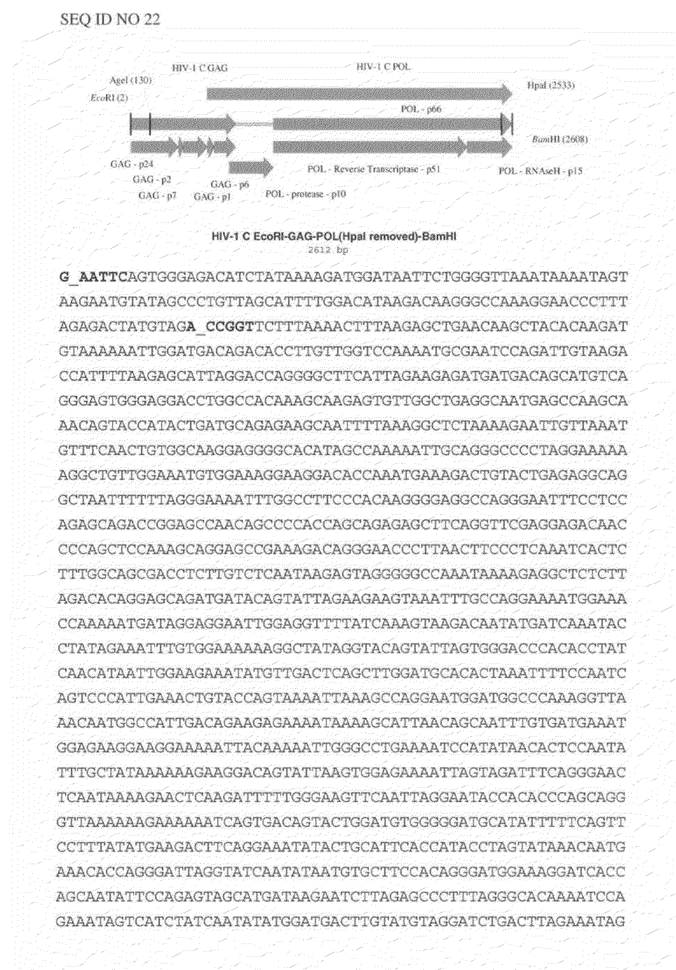

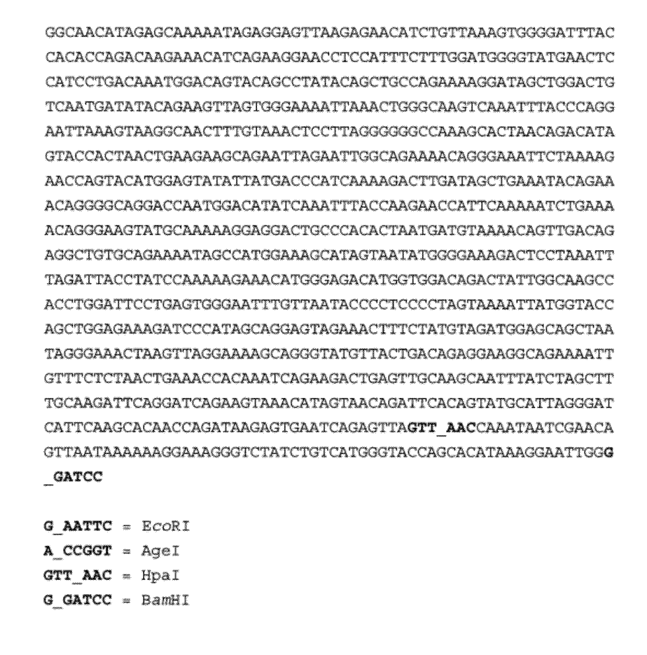

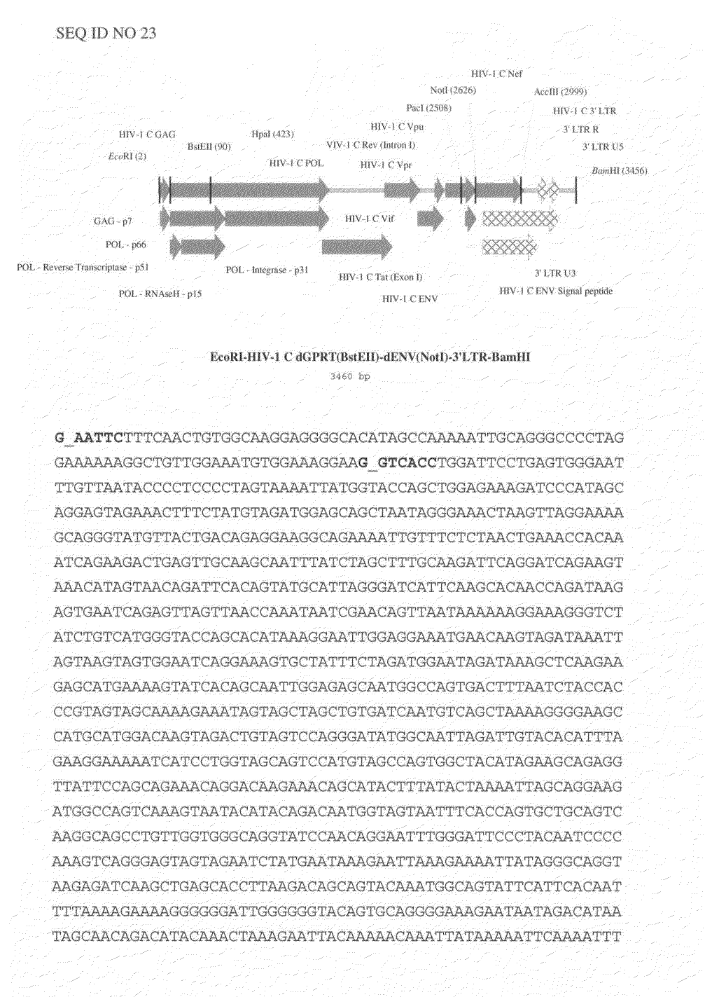

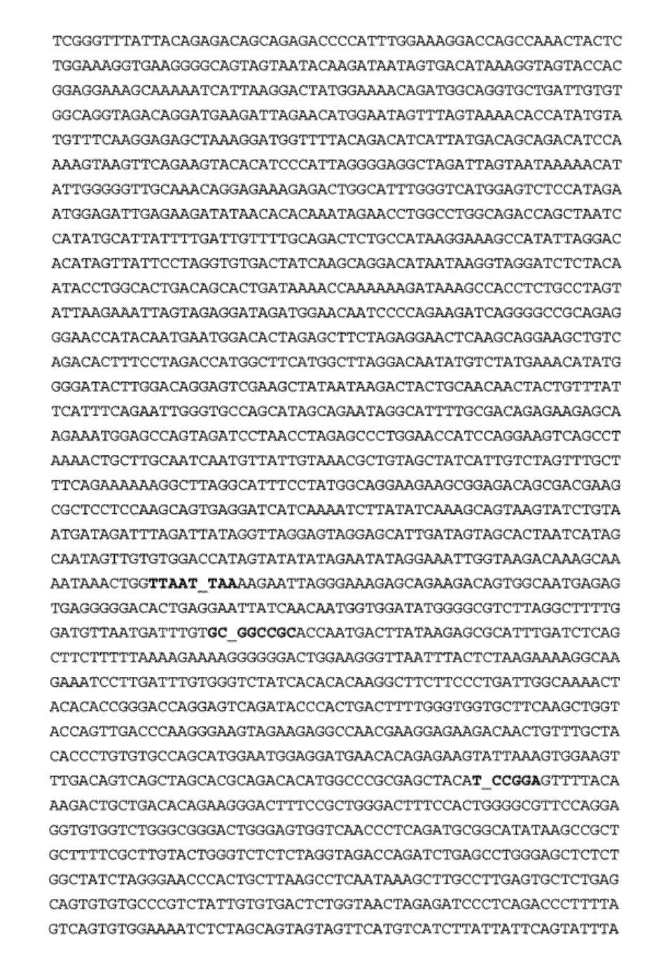

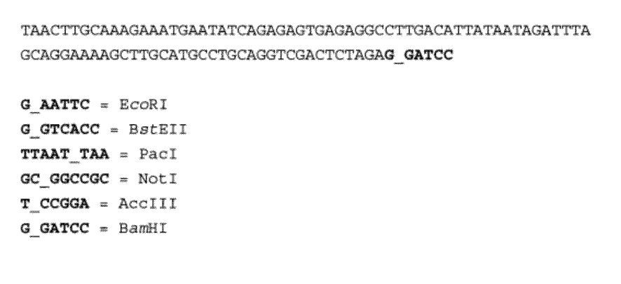

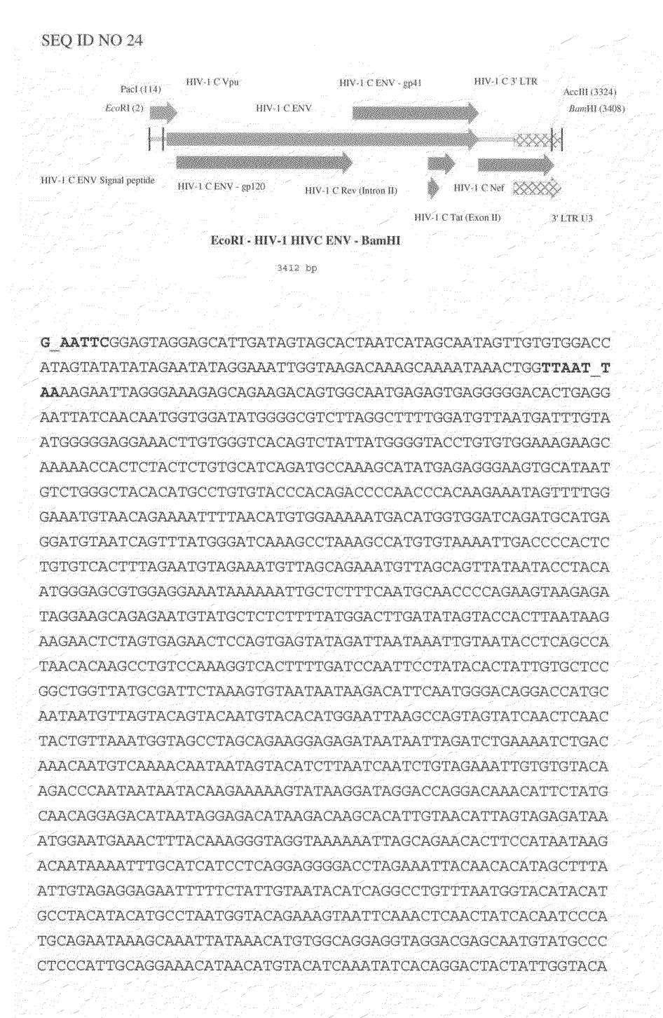

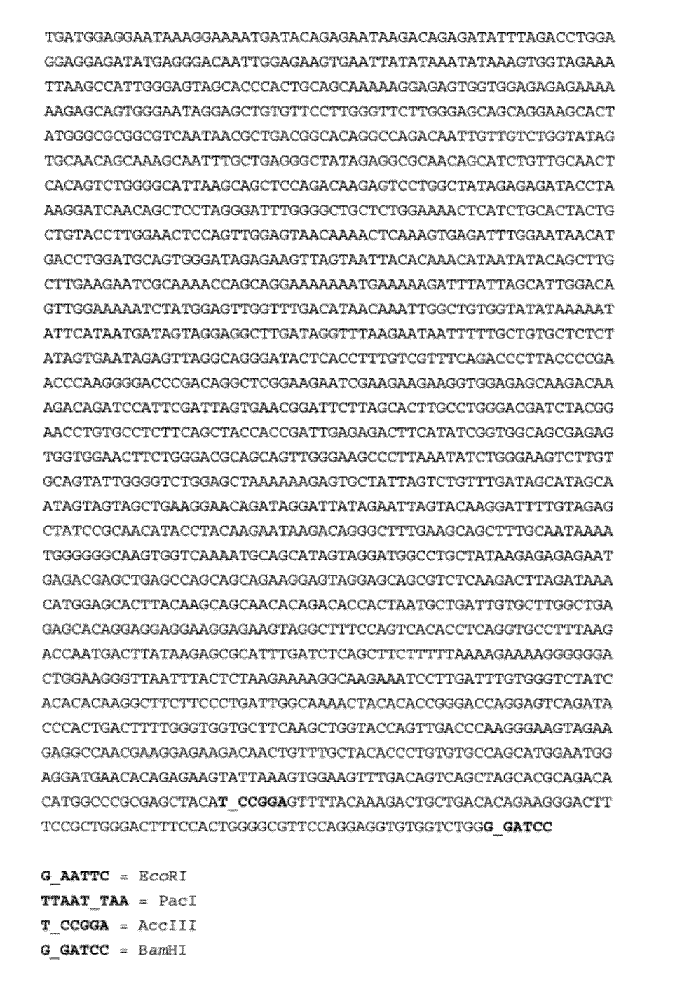

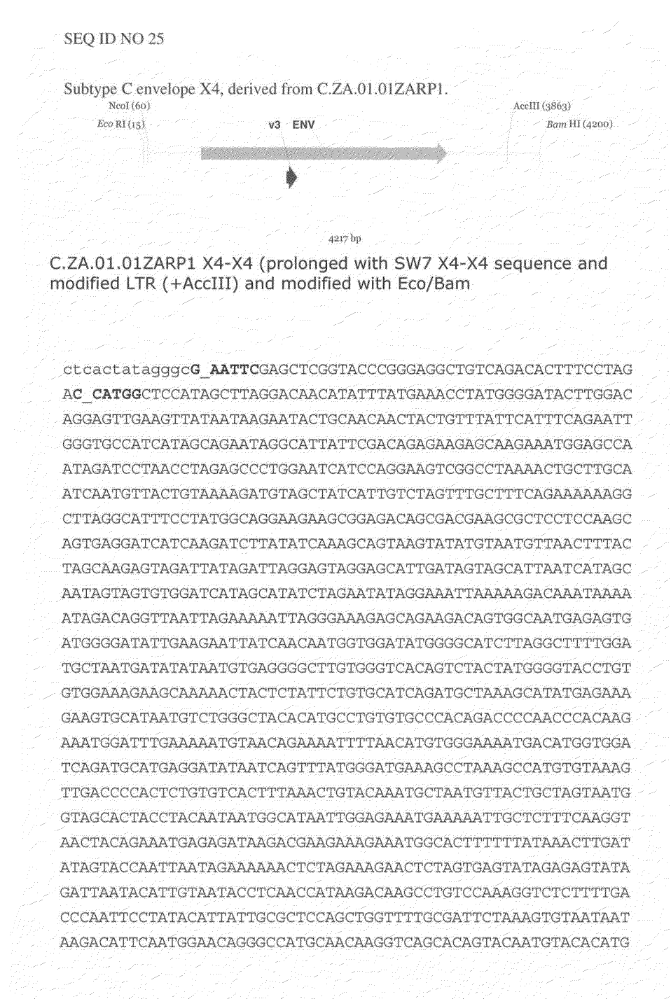

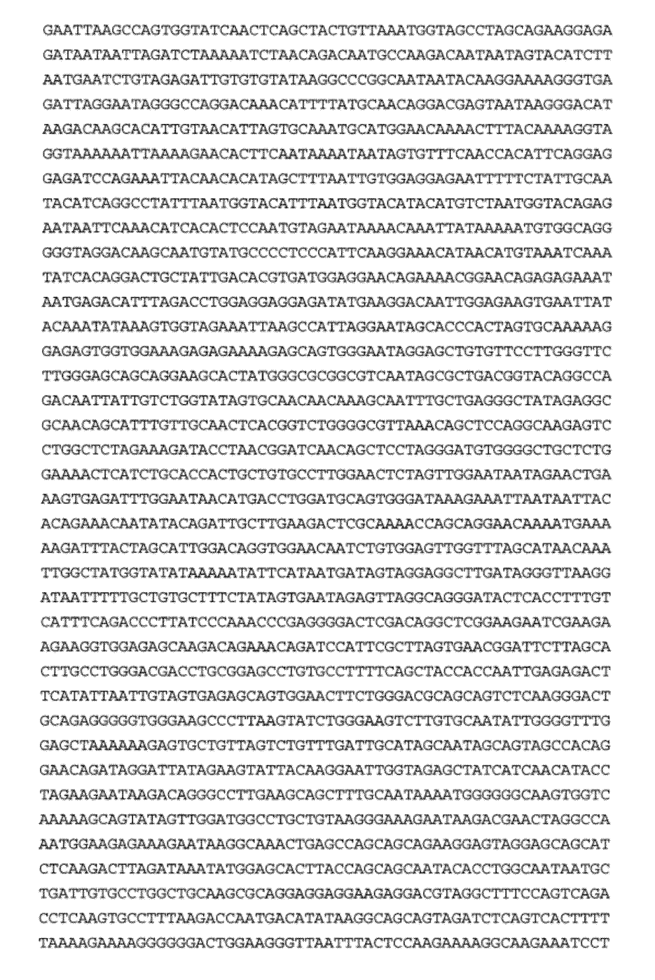

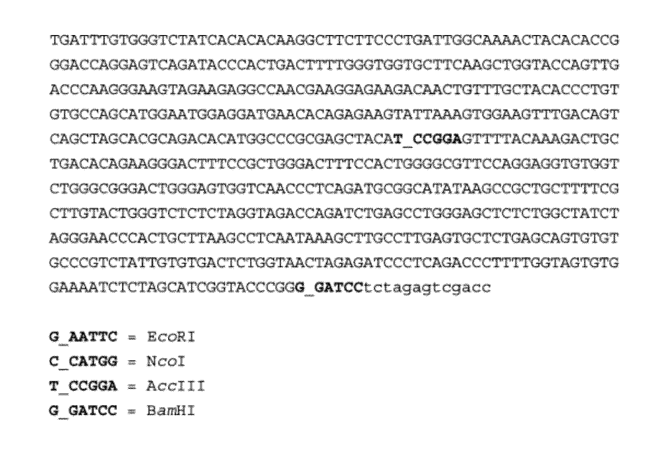

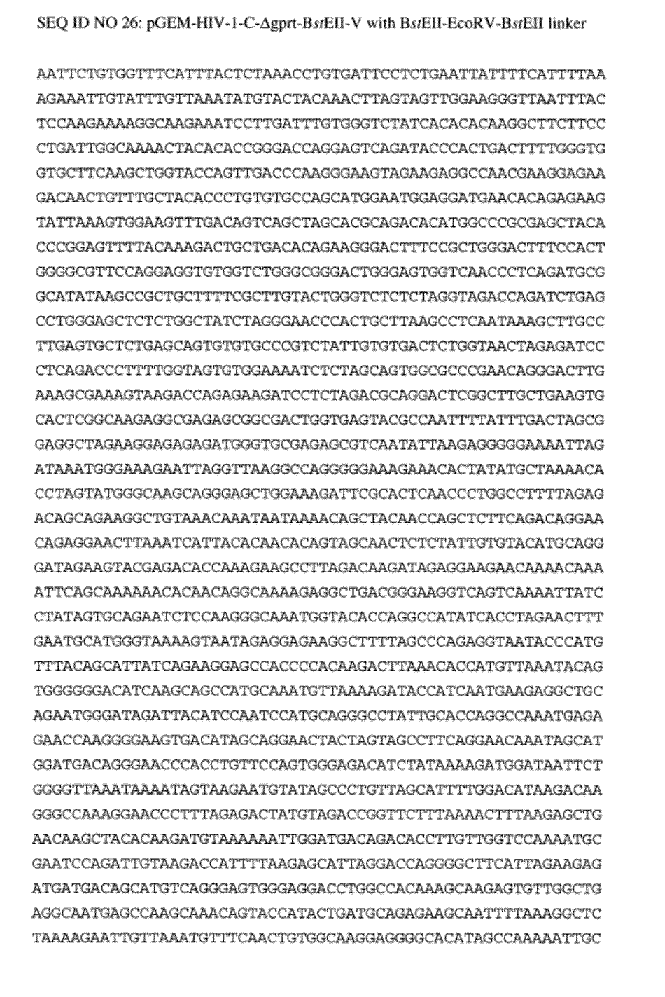

Fold-change values were calculated by dividing the IC50 values of the virus stocks harboring Resistance Associated Mutations (RAMs) by the IC50 values of the corresponding backbone with wild-type amplicon. Scatter plots showing the relationship between the FC values of the virus stocks carrying the GPRT subtype C amplicon in a subtype C backbone vs. FC values of the virus stocks carrying the GPRT subtype C amplicon in a subtype B backbone are shown in FIG. 3. The plots demonstrate an overall similarity in FC between subtype B and C recombinant virus stocks for all drug classes (FIG. 3-A, B, C, D). Correlations were high and very similar among the three drug classes: R²=0.88 (FIG. 3-A, all drug classes), 0.88 (FIG. 3-B, NRTI), 0.90 (FIG. 3-C, NNRTI) and 0.87 (FIG. 3-D, PI). The FC of the samples analyzed covered the entire resistance spectrum from virus fully susceptible to fully resistant to one or more drugs. The ratio FC_{subtype B}/FC_{subtype C} for most drugs was close to one (FIG. 3-E), indicating that the observed fold-change values of the GPRT amplicons in the subtype C backbone were very similar to the FC observed for that same amplicons in the subtype B backbone, but some differences were observed. The FC ratio was significantly different from 1 (p<0.05) for emtricitabine (FTC, p=0.031), nevirapine (NVP, p=0.043), etravirine (ETR, p=0.033), lopinavir (LPV, p=0.0041) and darunavir (DRV, p=0.002). The ratios for FTC (0.50) and ETR (0.67) suggest that, for these drugs, the FC in the subtype C backbone is higher than in the subtype B backbone, whereas for nevirapine (1.61), lopinavir (1.86) and darunavir (1.78) the opposite is true (FIG. 3-E).

### 6. Single Mutation Effects on FC

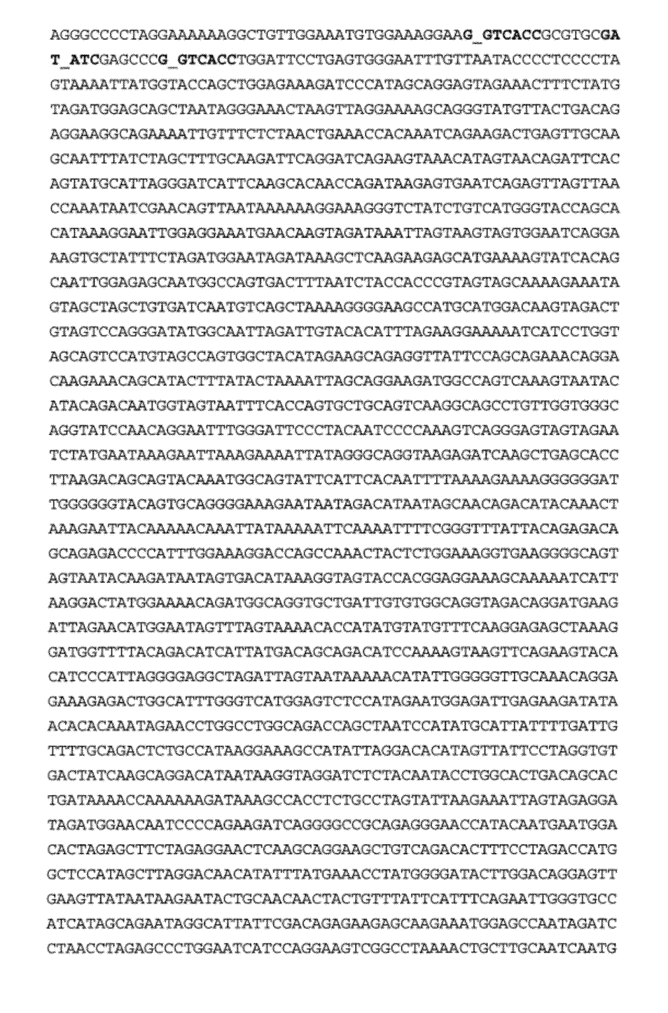

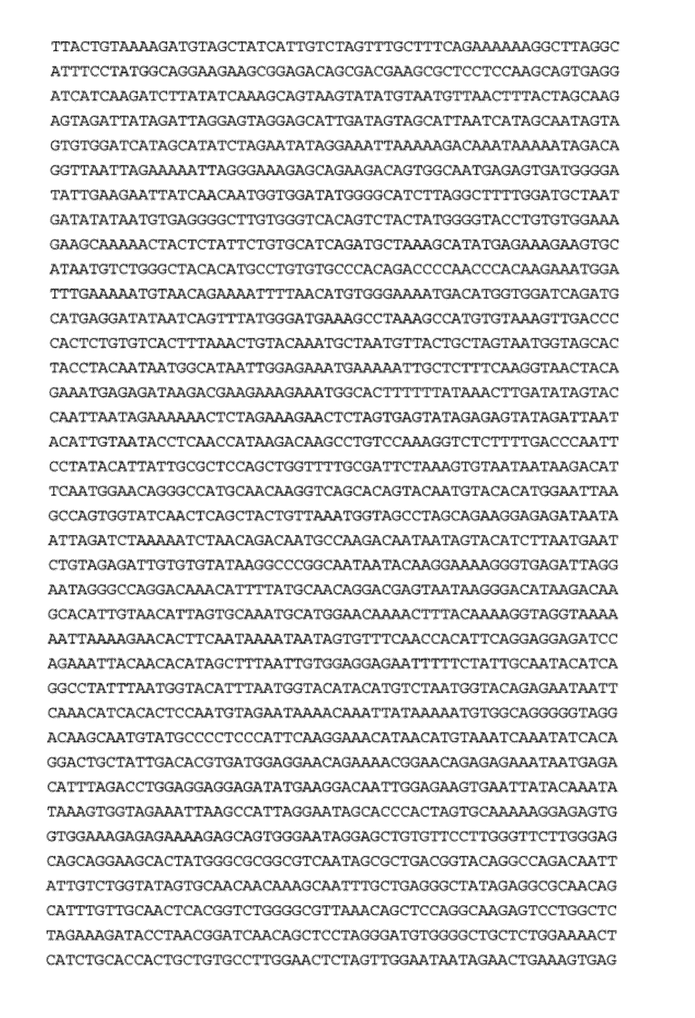

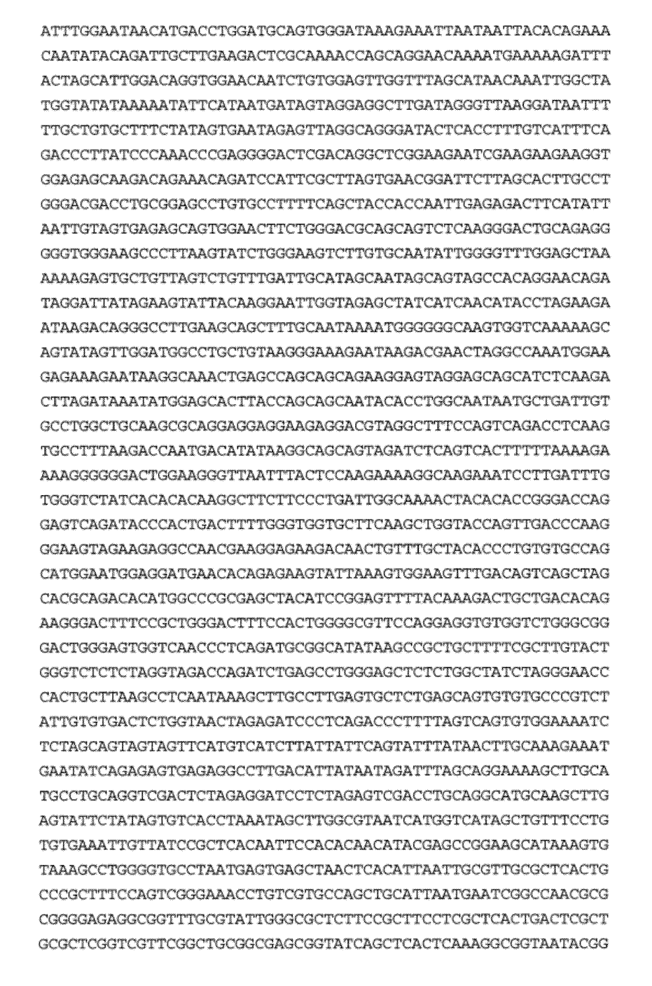

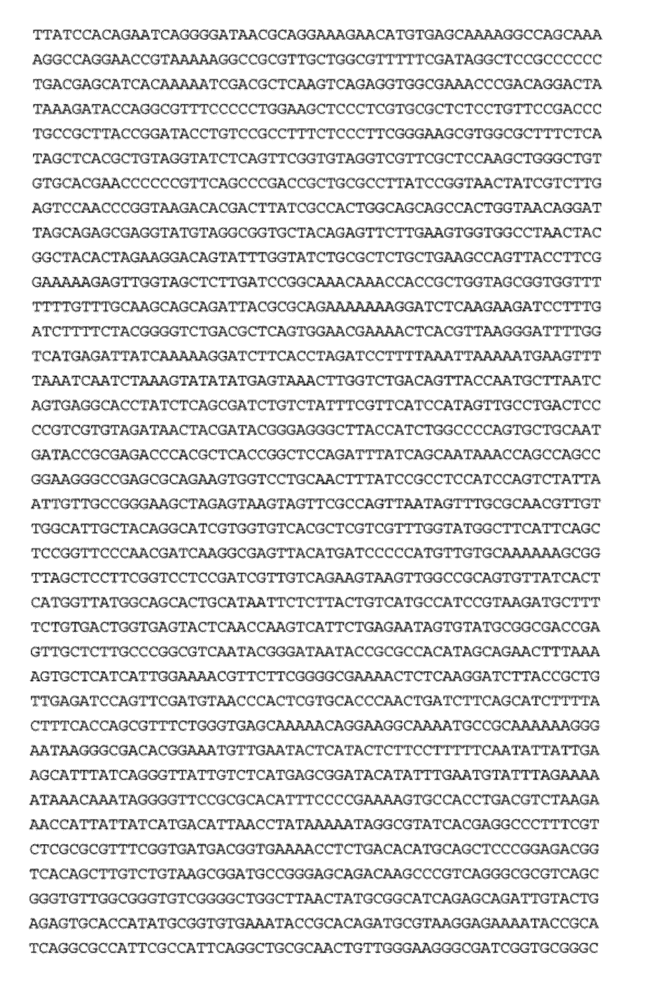

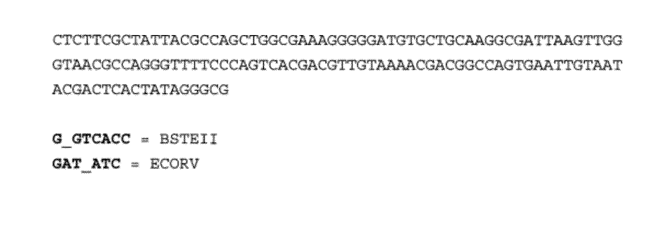

Clone 3 of Sample 3 enabled us to investigate the effect of a single RAM (M184V in RT) on the FC of viruses with the subtype C GPRT sequence inserted in the HIV-1 Subtype B and C backbones. In RT, a change at position 184 from methionine to valine results in an increase in FC for 3TC and FTC while it decreases the FC for AZT, d4T and TDF. This effect was observed with both types of backbone as shown in FIG. 4. The increase in FC is most pronounced and highly significant (p=<0.0001) for both 3TC and FTC and for both subtype backbones. The resensitizing effect for AZT was also highly significant (p<0.0001) in both subtype backbones while it was significant (p<0.05) only in the subtype C backbone for d4T (p_{C}=0.046; p_{B}=0.2576) and TDF (P_{C}=0.0267; p_{B}=0.1257). FCs for ddI and ABC were not significantly affected by the presence of 184V.

1. An in vitro method for designing a drug regimen for an HIV-1-C infected patient by determining the phenotypic susceptibility of HIV-1-C to at least one drug, comprising:
i) using at least one sample comprising HIV-1 RNA from a patient infected with HIV-1-C, wherein the sample comprises the HIV-1 gag-protease-reverse transcriptase coding region;
ii) reverse-transcribing and amplifying said HIV-1 RNA with primers specific for the HIV-1 gag-protease-reverse transcriptase (GPRT) coding region to obtain at least one amplicon comprising the HIV-1 GPRT coding region, wherein at least one primer is selected from
SEQ ID NO: 1
5′-GCCCCTAGGAAAAAGGGCTGTTGG-3′
SEQ ID NO: 2
5′-CATGAGAAATATCACAGTAATTGGAGAGCAATG-3′
SEQ ID NO: 3
5′-AATGTGGAAAGGAAGGACACCAAATGAAAG-3′
SEQ ID NO: 4
5′-CTCATAACCGTTCGGTGGACCTAAGGACT-3′
iii) generating a plasmid comprising a reference HIV-1-C sequence with a deletion of the HIV-1 GPRT coding region;
iv) preparing at least one recombinant virus by recombination or ligation between at least one amplicon obtained in step ii) and the plasmid comprising the reference HIV-1-C sequence with a deletion of the HIV-1 GPRT coding region obtained in step iii), and
v) monitoring at least one recombinant virus in the presence of at least one drug to determine the phenotypic susceptibility of HIV-1-C to at least one drug,
wherein said susceptibility is determined by the cytopathogenicity of said recombinant virus to cells or by determining the replicative capacity of said recombinant virus in the presence of at least one drug.

2. A method of constructing a genotypic and phenotypic database of GPRT sequences from HIV-1-C, comprising:
i) obtaining samples of HIV-1-C RNA comprising the GPRT coding region or a portion thereof
ii) reverse-transcribing and amplifying said HIV-1 RNA with primers selected from SEQ ID NO 1-4 specific for the GPRT coding region of the HIV-1-C genome to obtain an amplicon comprising the GPRT coding region or a portion thereof
iii) determining the nucleotide sequence of the amplicons or portions thereof Sequencing primers
SEQ ID NO: 5
5′-GAGAGCTTCAGGTTTGGGG-3′
SEQ ID NO: 6
5′-AATTGGGCCTGAAAATCC-3′
SEQ ID NO: 7
5′-CCTCCATTCCTTTGGATGGG-3′
SEQ ID NO: 8
5′-CACTCTTTGGCAACGACCC-3′
SEQ ID NO: 9
5′-CTCCCACTCAGGAATCC-3′
SEQ ID NO: 10
5′-CTTCCCAGAAGTCTTGAGTTC-3′
SEQ ID NO: 11
5′-GGGTCATAATACACTCCATG-3′
SEQ ID NO: 12
5′-GGAATATTGCTGGTGATCC-3′
SEQ ID NO: 13
5′-CAGACCAGAGCCAACAGCCCC-3′
SEQ ID NO: 14
5′-GGTACAGTATTAGTAGGACC-3′
SEQ ID NO: 15
5′-GTACTGGATGTGGGTGATGC-3′
SEQ ID NO: 16
5′-GTGGGAAAATTGAATTGGG-3′
SEQ ID NO: 17
5′-GTACTGTCCATTTATCAGG-3′
SEQ ID NO: 18
5′-CTAACTGGTACCATAATTTCACTAAGGGAGG-3′
SEQ ID NO: 19
5′-CATTGTTTAACTTTTGGGCC-3′
SEQ ID NO: 20
5′-GATAAAACCTCCAATTCC-3′
iv) preparing recombinant virus by homologous recombination or ligation between the amplicons and a plasmid comprising the wild-type HIV-1-C sequence with a deletion in the GPRT coding region of the HIV-1-C genome
v) determining the relative replicative capacity of the recombinant virus in the presence of anti-HIV drugs compared to HIV-1-C with a wild-type GPRT coding region sequence
vi) correlating the nucleotide sequence and relative replicative capacity in a data table.

3. A database comprising genotypic and phenotypic data of HIV-1-C GPRT coding regions, wherein the database further provides a correlation between genotypes and between genotypes and phenotypes, wherein the correlation is indicative of efficacy of a given drug regimen.

4. The vector pGEM-HIV-1-C-Δrt-BstEII-V having SEQ ID NO 26.

5. Use of the vector with SEQ ID NO 26 in the method of claim 1.

6. Use of the vector with SEQ ID NO 26 in the method of claim 2.
